SEQUENCE LISTING (59 sequences)

SEQ ID NO: 1 - protein, 432 aa, Artificial Sequence
  Antigen
MPIKHIHARQIYDSRGNPTVEVDLTTERGIFRAAVPSGASTGVHEALELRDKDSTWHGKSGLKAVKNVNDVLGPELVKKNLDPVKQEEIDDFMISLDGTDNKSKFGANSILGISMAVCKAGAAHKGVPLYRHIADLAGVKEVMMPVPAFNVINGGSHAGNKLAMQEFMILPTGAPSFTEAMRMGSEIYHHLKALIKKKYGLDATAVGDEGGFAPNFQANGEAIDLLVGAIEKAGYTGKIKIGMDVAASEFYKNGKYDLDFKNEESKEADWLTSEALGEMYKGFIKDAPVISIEDPYDQDDWEGWTALTSQTDIQIVGDDLTVTNPKRIQMAVDKKSCNCLLLKVNQIGSVTESIRAHNLAKSNGWGTMVSHRSGETEDCFIADLVVGLCTGQIKTGAPCRSERLSKYNQLLRIEEELGSNAKYVGDKFRMPF

SEQ ID NO: 2 - protein, 290 aa, Artificial Sequence
  Antigen
MMPVPAFNVINGGSHAGNKLAMQEFMILPTGAPSFTEAMRMGSEIYHHLKALIKKKYGLDATAVGDEGGFAPNFQANGEAIDLLVGAIEKAGYTGKIKIGMDVAASEFYKNGKYDLDFKNEESKEADWLTSEALGEMYKGFIKDAPVISIEDPYDQDDWEGRTALTSQTDIQIVGDDLTVTNPKRIQMAVDKKSCNCLLLKVNQIGSVTESIRAHNLAKSNGWGTMVSHRSGETEDCFIADLVVGLCTGQIKTGAPCRSERLSKYNQLLRIEEELGSNAKYVGDKFRMPF

SEQ ID NO: 3 - protein, 364 aa, Artificial Sequence
  Antigen
MGLEGIVPPGVITGDNLIKLFEYCRDHKVALPAFNCTSSSTINAVLQAARDIKSPVIVQFSNGGAAFMAGKGIKNDGQKASVLGAIAGAQHVRLMAKHYGVPVVLHSDHCAKKLLPWFDGMLEADEEYFKQNGEPLFSSHMLDLSEEFDEENISTCAKYFTRMTKMKMWLEMEIGITGGEEDGVDNTNVKAESLYTKPEQVYNVYKTLSEIGPMFSIAAAFGNVHGVYKAGNVVLSPHLLADHQKYIKEQINSPLDKPAFLVMHGGSGSTREEIAEAVSNGVIKMNIDTDTQWAYWDGLRKFYEEKKEYLQGQVGNPEGADKPNKKFYDPRVWVRAAEESMIKRANESFESLNAVNVLGDSWKH

SEQ ID NO: 4 - protein, 199 aa, Artificial Sequence
  Antigen
MSLQPTNDAPQFKAMAVVNKEFKEVSLKDYTGKYVVLFFYPLDFTFVCPTEIIAFGDRAADFRKIGCEVLACSTDSHFSHLHWINTPRKEGGLGDMDIPLIADKNMEISRAYGVLKEDDGVSFRGLFIIDGTQKLRQITINDLPVGRCVDETLRLVQAFQYTDVHGEVCPAGWKPGKKSMKPSKEGVSSYLADAEQSKK

SEQ ID NO: 5 - protein, 249 aa, Artificial Sequence
  Antigen
MGGGRKFFVGGNWKMNGDKKSIDGIVDFLSKGDLDPNCEVVVGASPCYLDYSRSKLPANIGVAAQNCYKVAKGAFTGEISPQMIKDVGCEWAILGHSERRNVFGESDELIGEKVAFALESGLKIIPCIGEKLDERESGKTEEVCFKQLKAISDKVSDWDLVVLAYEPVWAIGTGKTATPAQAQETHLALRKWLKENVSEEVSQKVRILYGGSVSAGNCKELGTQPDIDGFLVGGASLKPDFVQIINATK

SEQ ID NO: 6 - protein, 172 aa, Artificial Sequence
  Antigen
MKIFKDVFSGDELFSDTYKFKLLDDCLYEVYGKYVTRTEGDVVLDGANASAEEAMDDCDSSSTSGVDVVLNHRLVETGFGSKKDYTVYLKDYMKKVVTYLEENGKQAEVDTFKTNINKVMKELLPRFKDLQFYTGETMDPEAMIIMLEYKEVDGKDIPVLYFFKHGLNEEKF

SEQ ID NO: 7 - DNA, 1299 bp, Artificial Sequence
  Antigen
atgcctattaaacacattcatgcacgtcaaatctacgactctcgtggtaaccctacagtggaggtggatctcaccactgagcgagggattttccgcgctgccgtccccagtggagcttccacaggggttcatgaggccctggaactgcgcgacaaggactctacctggcacgggaagagtggtctcaaggctgtgaagaatgtgaacgacgtccttgggcccgagttggtgaagaagaaccttgaccccgtgaagcaagaggagatcgatgatttcatgatcagcctcgacgggacggataacaagagcaaatttggggctaattctattttgggaatctcgatggctgtgtgcaaggctggtgccgcccacaagggtgttcccctctaccgccatatcgctgacttggcgggtgtgaaggaagtgatgatgccggtgcccgcatttaatgtcattaacggaggttctcatgctggaaataagttggcgatgcaagaattcatgatccttccaactggagctccctccttcactgaagccatgaggatgggatctgaaatctatcaccatctcaaggctcttatcaagaagaagtacgggttggatgctacagccgttggagatgagggtggctttgctcccaacttccaagccaacggcgaggctatcgaccttcttgttggagccattgaaaaggctggatacactggaaaaatcaagatcggaatggatgttgctgcttcagaattttacaaaaatggaaagtacgatttagatttcaaaaatgaagaatccaaagaggccgattggctaacttccgaggctcttggtgaaatgtacaaaggattcatcaaggatgcacctgtcatttccattgaagatccctacgatcaagatgattgggagggatggactgcattgacatcacaaactgacattcagattgtcggagatgatctcacagtcacaaaccccaagcgtattcaaatggctgttgacaagaaatcttgcaactgcctcctcttgaaagtaaatcaaattggttcagtaactgaatctattcgggcccacaatcttgctaagagcaacggctggggtaccatggtctctcatagatctggtgagacagaggattgtttcatcgctgatctcgtcgttggtctctgcactggtcaaatcaagactggagctccttgcagatccgaacgtttgtctaaatacaatcaattgttgcgtattgaagaggagttgggatccaacgctaaatatgtcggtgacaagttcagaatgcccttttaa

SEQ ID NO: 8 - DNA, 1296 bp, Artificial Sequence
  Antigen
atgccgattaaacacatccatgcccgccaaatctatgactcccgtggtaacccgaccgttgaagttgacctgaccaccgaacgtggcatttttcgtgccgcggtgccgagcggtgcatctacgggtgttcatgaagctctggaactgcgcgataaagactcaacctggcacggcaaatcgggtctgaaagcggtcaaaaacgtgaatgatgttctgggcccggaactggtgaagaaaaacctggacccggtcaaacaggaagaaattgatgactttatgatcagcctggatggtaccgacaacaaatctaaattcggcgcaaatagtattctgggtatctccatggcagtctgtaaagctggcgcagctcataaaggtgtgccgctgtatcgtcacattgcggatctggccggcgtcaaagaagtgatgatgccggttccggccttcaacgtcattaatggcggtagccatgcaggtaataaactggctatgcaggaatttatgattctgccgaccggtgccccgtcattcaccgaagccatgcgcatgggttcggaaatttatcatcacctgaaagcgctgattaagaaaaaatacggcctggatgcaacggctgttggtgacgaaggcggttttgccccgaacttccaagcgaatggcgaagccattgatctgctggttggtgcaatcgaaaaagctggctacaccggtaaaattaaaatcggcatggatgtcgcggcctccgaattctacaaaaacggtaaatacgatctggacttcaaaaatgaagaaagtaaagaagcggattggctgaccagcgaagccctgggcgaaatgtacaaaggtttcatcaaagatgccccggtgattagcatcgaagatccgtacgaccaggatgactgggaaggctggaccgcactgacgtctcagaccgatattcaaatcgtgggtgatgacctgaccgttacgaacccgaaacgtatccagatggcggttgataaaaaatcttgcaactgtctgctgctgaaagtcaatcaaattggctcagtgaccgaatcgatccgtgcgcataacctggccaaatctaatggctggggtacgatggtgtctcaccgctccggcgaaaccgaagattgcttcattgcagacctggtggttggcctgtgtacgggtcagatcaaaaccggtgctccgtgccgtagcgaacgcctgtctaaatataatcaactgctgcgcatcgaagaagaactgggtagcaatgcgaaatatgtgggtgataaattccgtatgccgttt

SEQ ID NO: 9 - DNA, 1095 bp, Artificial Sequence
  Antigen
atgggtcttgaaggaattgttccccctggtgtcatcactggagacaatcttattaagttgttcgaatactgcagagaccataaagttgctctccctgctttcaactgcacgtcttcttcaaccatcaatgcagttttgcaagcagcacgggacattaaatcccctgtgattgttcaattttccaatggtggagctgcttttatggccggcaaaggcatcaaaaatgacggtcaaaaggctagtgtccttggtgcaattgctggggctcaacatgttcgtttaatggcaaagcactatggtgttcctgtagttcttcactctgatcactgtgctaaaaaactcctcccatggtttgatggaatgcttgaagctgatgaagagtatttcaaacaaaatggtgaacctcttttctccagtcacatgcttgatctctcggaggagtttgatgaagaaaatatttccacttgtgcaaaatattttactcgcatgactaaaatgaaaatgtggttagaaatggaaattggaatcactgggggcgaagaggatggtgttgacaataccaatgtgaaagcggagtctctttacaccaaacccgaacaagtttacaacgtgtacaaaacactcagcgaaattggaccaatgttttccattgctgccgcttttggaaacgtacatggtgtatacaaggcaggtaacgttgttctttccccacatttgttggctgatcatcaaaaatacatcaaggagcaaattaactccccacttgataaacccgccttccttgtcatgcacggaggctccggctccaccagagaagaaattgctgaagcagtaagcaacggtgtgatcaaaatgaatattgatacggatactcaatgggcttactgggatggtctcagaaagttttatgaagaaaagaaggagtatcttcaaggacaggttggaaatccagaaggcgctgacaagccaaacaaaaagttttacgatccacgagtttgggttcgtgctgctgaggagtctatgattaagagagccaatgaatcctttgaatcattaaacgctgtgaatgtccttggtgactcctggaaacactaa

SEQ ID NO: 10 - DNA, 1092 bp, Artificial Sequence
  Antigen
atgggtctggaaggcatcgttccgccgggtgtcattacgggtgataacctgattaaactgttcgaatactgccgcgaccacaaagtggcactgccggcttttaactgcaccagctctagtacgattaatgcagtgctgcaggcggcccgtgatattaaatctccggttatcgtccaatttagtaacggcggtgcagctttcatggcgggcaaaggtattaaaaatgatggccagaaagcctccgttctgggcgccatcgcaggtgctcaacatgttcgcctgatggccaaacactatggtgtcccggtggttctgcattctgatcactgcgcgaaaaaactgctgccgtggttcgatggcatgctggaagccgacgaagaatactttaaacagaacggtgaaccgctgttctcctcacacatgctggatctgtcggaagaatttgacgaagaaaatatcagcacctgtgcgaaatatttcacccgtatgacgaaaatgaaaatgtggctggaaatggaaattggcatcacgggcggtgaagaagatggtgtcgacaacaccaatgtgaaagccgaaagcctgtatacgaaaccggaacaggtctataacgtgtacaaaaccctgtccgaaattggcccgatgttttcaatcgcggccgcattcggcaacgttcatggtgtctataaagccggtaatgtcgtgctgtctccgcatctgctggctgatcaccagaaatacatcaaagaacaaatcaacagtccgctggacaaaccggcgtttctggtgatgcatggcggttcgggtagcacccgtgaagaaattgcggaagccgtgagcaacggtgttattaaaatgaatatcgataccgacacgcagtgggcatattgggatggcctgcgcaaattctacgaagaaaagaaagaatacctgcagggccaagttggtaacccggaaggtgctgataaaccgaataaaaaattctatgacccgcgtgtgtgggttcgtgctgccgaagaaagtatgatcaaacgcgctaacgaatcctttgaatccctgaacgcagtgaatgtgctgggtgacagttggaaacac

SEQ ID NO: 11 - DNA, 600 bp, Artificial Sequence
  Antigen
atgagtcttcaaccaacgaatgatgctcctcaattcaaggctatggccgttgtgaacaaggaattcaaggaggtgtcactcaaggactataccggcaaatacgtggttctctttttctaccccttggactttacctttgtttgccccacagaaatcattgcctttggagatcgggctgcagatttccgtaaaattggatgtgaggtccttgcctgctccactgactcccatttttctcatctccactggatcaacactcctcgtaaggagggaggacttggggacatggacattcccctcattgcggataagaacatggaaatttctagagcctatggcgtgctcaaggaagacgatggagtgtccttcagaggacttttcatcattgacggcactcagaaactccgtcaaatcaccatcaatgatcttcctgtcggaagatgcgtagacgaaaccttaagacttgtacaagccttccaatacacggacgtgcatggcgaggtttgccctgcgggatggaagccaggaaagaagtctatgaagcccagcaaggaaggtgtctcatcttacctcgcagatgctgaacaatcaaagaaataa

SEQ ID NO: 12 - DNA, 597 bp, Artificial Sequence
  Antigen
atgtcactgcaaccgacgaacgacgccccgcaattcaaagcaatggcagtggttaacaaagaattcaaagaagtttcgctgaaagattacaccggcaaatacgtcgtgctgtttttctatccgctggactttaccttcgtctgcccgacggaaattatcgcatttggcgatcgtgcggccgacttccgcaaaattggttgcgaagtgctggcttgtagcaccgattctcatttcagtcatctgcactggatcaacacgccgcgtaaagaaggcggtctgggcgatatggacattccgctgatcgcagataaaaatatggaaatttcccgcgcttatggtgtcctgaaagaagatgacggcgtgtcatttcgtggtctgttcattatcgacggcacccagaaactgcgccaaattacgatcaatgatctgccggttggtcgttgcgtcgacgaaaccctgcgcctggttcaggcgtttcaatacacggatgtgcacggtgaagtttgtccggccggctggaaaccgggtaaaaaatctatgaaaccgtcaaaagaaggcgtgtcgtcctacctggcagatgctgaacaatccaaaaaa

SEQ ID NO: 13 - DNA, 750 bp, Artificial Sequence
  Antigen
atgggtggaggaagaaaatttttcgttggtggaaactggaaaatgaatggagacaagaaatctattgatggaatcgtagattttttgagcaagggggatttggacccaaattgtgaggttgttgttggagcctcaccctgctatttggactattcccgttctaaacttcctgccaatatcggagtggctgcacaaaattgttataaggtggccaaaggagcatttaccggagaaatcagtcctcaaatgattaaagatgttggttgtgaatgggcgattcttggtcattcagagcgtagaaatgtctttggggaatctgatgagctcattggcgaaaaggttgcttttgcacttgagtctggtctcaaaattattccatgcattggagaaaaattagacgaacgtgaatctgggaagactgaggaggtctgctttaagcaacttaaagccatttctgacaaagtatctgattgggatcttgtcgtcttagcttatgaaccagtttgggccattggaactggcaaaacagctacacctgctcaggctcaagaaacacatcttgctcttcgtaaatggctaaaggagaacgtttctgaggaagtttcacaaaaagtgcgaatcctctatggaggttccgtgagtgctggtaattgcaaggaacttggcactcagcctgatattgacggcttccttgttggaggagcctctctcaaacctgactttgttcaaatcatcaacgctactaagtaa

SEQ ID NO: 14 - DNA, 747 bp, Artificial Sequence
  Antigen
atgggcggcggtcgcaaattctttgtcggcggcaactggaaaatgaacggcgataaaaaatctatcgatggtatcgtggattttctgagcaaaggcgatctggatccgaattgcgaagtggttgtgggtgcgagcccgtgttatctggattacagccgttctaaactgccggcaaacattggtgtggccgcacagaattgctataaagttgcgaaaggcgccttcaccggtgaaattagcccgcagatgatcaaagatgttggctgtgaatgggcaattctgggtcattctgaacgtcgcaacgtgtttggcgaaagtgatgaactgatcggtgaaaaagttgcattcgcgctggaaagcggcctgaaaattatcccgtgcatcggtgaaaaactggatgaacgcgaatctggtaaaacggaagaagtgtgttttaaacagctgaaagccatttctgataaagttagtgattgggatctggttgtgctggcgtatgaaccggtgtgggcgattggtaccggtaaaaccgcaacgccggcacaggcacaggaaacccacctggcactgcgtaaatggctgaaagaaaacgttagcgaagaagtgtctcagaaagttcgcattctgtacggcggtagtgttagcgcgggcaattgcaaagaactgggtacccagccggatatcgatggcttcctggtgggtggtgcttccctgaaaccggactttgtgcagattatcaacgctacgaaa

SEQ ID NO: 15 - DNA, 519 bp, Artificial Sequence
  Antigen
atgaagatctttaaggacgtattttctggagatgaattattttccgacacctacaagttcaagttgttggatgattgcttgtacgaggtgtatggaaagtatgtcacacggactgaaggagatgtggttcttgatggagccaacgcatctgctgaagaggccatggatgactgtgattcctcttccacctctggtgtcgatgttgtccttaaccaccgtctggtcgaaactgggttcggttccaagaaggactacaccgtataccttaaggactacatgaagaaggtagtgacatatttagaagaaaatggcaaacaagccgaagtagataccttcaagaccaacatcaacaaggtcatgaaggaacttttaccacggtttaaggatcttcaattctatactggagaaacgatggaccctgaggccatgatcatcatgcttgaatacaaggaagttgatggaaaggatattcccgtcctctacttttttaaacatggattaaatgaagaaaaattttaa

SEQ ID NO: 16 - DNA, 516 bp, Artificial Sequence
  Antigen
atgaaaatcttcaaagacgtgtttagcggcgacgaactgttctcggatacctacaaatttaaactgctggatgattgcctgtatgaagtgtacggcaaatatgttacccgtacggaaggcgatgtggttctggatggtgcgaacgccagcgcagaagaagcgatggatgattgtgatagctctagtacctctggtgtggatgtggttctgaatcatcgcctggttgaaaccggctttggtagcaagaaagattacacggtgtatctgaaagattacatgaagaaagtggttacgtatctggaagaaaacggcaaacaggcggaagtggataccttcaaaacgaacatcaacaaagttatgaaagaactgctgccgcgttttaaagatctgcagttctacaccggtgaaacgatggatccggaagccatgattatcatgctggaatataaagaagttgatggcaaagacattccggtgctgtacttcttcaaacacggcctgaacgaagaaaaattc

SEQ ID NO: 17 - RNA, 1630 nt, Artificial Sequence
  Enolase mRNA
gctccgattcacttcttatttctcaacgctcatcgatactttataaggctcaaattcaaaatgcctattaaacacattcatgcacgtcaaatctacgactctcgtggtaaccctacagtggaggtggatctcaccactgagcgagggattttccgcgctgccgtccccagtggagcttccacaggggttcatgaggccctggaactgcgcgacaaggactctacctggcacgggaagagtggtctcaaggctgtgaagaatgtgaacgacgtccttgggcccgagttggtgaagaagaaccttgaccccgtgaagcaagaggagatcgatgatttcatgatcagcctcgacgggacggataacaagagcaaatttggggctaattctattttgggaatctcgatggctgtgtgcaaggctggtgccgcccacaagggtgttcccctctaccgccatatcgctgacttggcgggtgtgaaggaagtgatgatgccggtgcccgcatttaatgtcattaacggaggttctcatgctggaaataagttggcgatgcaagaattcatgatccttccaactggagctccctccttcactgaagccatgaggatgggatctgaaatctatcaccatctcaaggctcttatcaagaagaagtacgggttggatgctacagccgttggagatgagggtggctttgctcccaacttccaagccaacggcgaggctatcgaccttcttgttggagccattgaaaaggctggatacactggaaaaatcaagatcggaatggatgttgctgcttcagaattttacaaaaatggaaagtacgatttagatttcaaaaatgaagaatccaaagaggccgattggctaacttccgaggctcttggtgaaatgtacaaaggattcatcaaggatgcacctgtcatttccattgaagatccctacgatcaagatgattgggagggatggactgcattgacatcacaaactgacattcagattgtcggagatgatctcacagtcacaaaccccaagcgtattcaaatggctgttgacaagaaatcttgcaactgcctcctcttgaaagtaaatcaaattggttcagtaactgaatctattcgggcccacaatcttgctaagagcaacggctggggtaccatggtctctcatagatctggtgagacagaggattgtttcatcgctgatctcgtcgttggtctctgcactggtcaaatcaagactggagctccttgcagatccgaacgtttgtctaaatacaatcaattgttgcgtattgaagaggagttgggatccaacgctaaatatgtcggtgacaagttcagaatgcccttttaatgatctaaagggttgtttcttcattgaagaaagttcatttctatagtcacaataaattatttcatggttttacaagaaattcacaggacgaaaaaacaaaaatcttaatttattgaattatttctatatgtattacacgcgtactctaagtaaaaccttataaaggaatataattgtaatataatttattgtaatattttttttttcatatttaatttatattaagggttgccatttaaatatataaattccccgttggtaaaaaaaaaaa

SEQ ID NO: 18 - RNA, 1539 nt, Artificial Sequence
  FBP aldolase mRNA
ggggggagttagtataagagatcgacaggctctgttcgcaacacttgttcctaaaggcaaattatcttaaatcttaaaaatgggtcttgaaggaattgttccccctggtgtcatcactggagacaatcttattaagttgttcgaatactgtagagaccataaagttgctctccctgctttcaactgcacgtcttcttcaaccatcaatgcagttttgcaagcagcacgggacattaaatcccctgtgattgttcaattttccaatggtggagctgcttttatggccggcaaaggcatcaaaaatgacggtcaaaaggctagtgtccttggtgcaattgctggggctcaacatgttcgtttaatggcaaagcactatggtgttcctgtagttcttcactctgatcactgtgctaaaaaactcctcccatggtttgatggaatgcttgaagctgatgaagagtatttcaaacaaaatggtgaacctcttttctccagtcacatgcttgatctctcggaggagtttgatgaagaaaatatttccacttgtgcaaaatattttactcgcatgactaaaatgaaaatgtggttagaaatggaaattggaatcactgggggcgaagaggatggtgttgacaataccaatgtgaaagcggagtctctttacaccaaacccgaacaagtttacaacgtgtacaaaacactcagcgaaattggaccaatgttttccattgctgccgcttttggaaacgtacatggtgtatacaaggcaggtaacgttgttctttccccacatttgttggctgatcatcaaaaatacatcaaggagcaaattaactccccacttgataaacccgccttccttgtcatgcacggaggctccggctccaccagagaagaaattgctgaagcagtaagcaacggtgtgatcaaaatgaatattgatacggatactcaatgggcttactgggatggtctcagaaagttttatgaagaaaagaaggagtatcttcaaggacaggttggaaatccagaaggcgctgacaagccaaacaaaaagttttacgatccacgagtttgggttcgtgctgctgaggagtctatgattaagagagccaatgaatcctttgaatcattaaacgctgtgaatgtccttggtgactcctggaaacactaaatacttattattggatattcagaatgttttaatttctattttggaactccgaacttactagtaatttatttctcttttaaaaaatgaatcagtatatttattattctgtttataaaattaagttattgttaatttccttaaatttatttatcaaaaattagaaattgttatacatgaaacattgacataaatctaaaattgaaacattttatgattttgatgtttataaatgctagataagaagtcataaataaatgtataataaattaaacttctttcgtgattaattaacttgctaattaatgcataattttcatttttttgaagatatgcgctaaaaaattattcaataaaaattaaaatag

SEQ ID NO: 19 - RNA, 888 nt, Artificial Sequence
  PRX-2 mRNA
gggggagtcttatatctgctaccggcaagtgaactacctctgtcatctctctttgtaatatccgactaagtaacaaaatgagtcttcaaccaacgaatgatgctcctcaattcaaggctatggccgttgtgaacaaggaattcaaggaggtgtcactcaaggactataccggcaaatacgtggttctctttttctaccccttggactttacctttgtttgccccacagaaatcattgcctttggagatcgggctgcagatttccgtaaaattggatgtgaggtccttgcctgctccactgactcccatttttctcatctccactggatcaacactcctcgtaaggagggaggacttggggacatggacattcccctcattgcggataagaacatggaaatttctagagcctatggcgtgctcaaggaagacgatggagtgtccttcagaggacttttcatcattgacggcactcagaaactccgtcaaatcacaatcaatgatcttcctgtcggaagatgcgtagacgaaaccttaagacttgtacaagccttccaatacacagacgtgcatggcgaggtttgccctgcgggatggaagccaggaaagaagtctatgaagcccagcaaggaaggtgtctcatcttacctcgcagatgctgaacaatcaaagaaataatacagaagatctcccctgtagttattagtttccataccaattctctcttttaattcattcgattggacactgttaccatgttccactttttaattgtacctggtcagtcagtgcccaaggtcattgattgattaagtctatcaaatatttatgtattccccggtgtactaatagtttttaagatataaaatatacgactttttaatatatt

SEQ ID NO: 20 - RNA, 1021 nt, Artificial Sequence
  TIM mRNA
gggggagttataaagcactactcgattgctaagtacttcgcgaggttcctactaattgtaatatagttgaaaaaatacattcaaaaatgggtggaggaagaaaatttttcgttggtggaaactggaaaatgaatggagacaagaaatctattgatggaatcgtagattttttgagcaagggggatttggacccaaattgtgaggttgttgttggagcctcaccctgctatttggactattcccgttctaaacttcctgccaatatcggagtggctgcacaaaattgttataaggtggccaaaggagcatttaccggagaaatcagtcctcaaatgattaaagatgttggttgtgaatgggcgattcttggtcattcagagcgtagaaatgtctttggggaatctgatgagctcattggcgaaaaggttgcttttgcacttgagtctggtctcaaaattattccatgcattggagaaaaattagacgaacgtgaatctgggaagactgaggaggtctgctttaagcaacttaaagccatttctgacaaagtatctgattgggatcttgtcgtcttagcttatgaaccagtttgggccattggaactggcaaaacagctacacctgctcaggctcaagaaacacatcttgctcttcgtaaatggctaaaggagaacgtttctgaggaagtttcacaaaaagtgcgaatcctctatggaggttccgtgagtgctggtaattgcaaggaacttggcactcagcctgatattgacggcttccttgttggaggagcctctctcaaacctgactttgttcaaatcatcaacgctactaagtaaacaaaatactggatattcgactcttctataatagtcttatcatctctttaatgctctcactcattatttgataaataacgaggttaaaatattatttatttgattaaacgtaatctaacgtaatacatatatattaattttcacgaatgcagaaaaaaaattattgcataaatacgtattttaca

SEQ ID NO: 21 - RNA, 846 nt, Artificial Sequence
  TCTP mRNA
gaggttgtcggctttcaaggaccactcaattcctccctagttctaattcactttcactccggactcttcccgtaaacactcctgccttatacaaaatgaagatctttaaggacgtattttctggagatgaattattttccgacacctacaagttcaagttgttggatgattgcttgtacgaggtgtatggaaagtatgtcacacggactgaaggagatgtggttcttgatggagccaacgcatctgctgaagaggccatggatgactgtgattcctcttccacctctggtgtcgatgttgtccttaaccaccgtctggtcgaaactgggttcggttccaagaaggactacaccgtataccttaaggactacatgaagaaggtagtgacatatttagaagaaaatggcaaacaagccgaagtagataccttcaagaccaacatcaacaaggtcatgaaggaacttttaccacggtttaaggatcttcaattctatactggagaaacgatggaccctgaggccatgatcatcatgcttgaatacaaggaagttgatggaaaggatattcccgtcctctacttttttaaacatggattaaatgaagaaaaattttaaacattagtgtcatcattcatctcaatttcttataaatgtttatatctacaatatattttatatagataaaaaagaatttccgttgacaataatatgcgaactacctaattaaattatgttgtattcatatttctaatgcgatttttgggaaatttctcgttataactaaattccatttttaacgtacacgtctgtatatgaatatatgtaaagtgttatttacttgtaagac

SEQ ID NO: 22 - DNA, 1338 bp, Artificial Sequence
  Enolase expression DNA sequence
atgcatcatcaccatcaccacgaaaacctgtattttcagggcatgccgattaaacacatccatgcccgccaaatctatgactcccgtggtaacccgaccgttgaagttgacctgaccaccgaacgtggcatttttcgtgccgcggtgccgagcggtgcatctacgggtgttcatgaagctctggaactgcgcgataaagactcaacctggcacggcaaatcgggtctgaaagcggtcaaaaacgtgaatgatgttctgggcccggaactggtgaagaaaaacctggacccggtcaaacaggaagaaattgatgactttatgatcagcctggatggtaccgacaacaaatctaaattcggcgcaaatagtattctgggtatctccatggcagtctgtaaagctggcgcagctcataaaggtgtgccgctgtatcgtcacattgcggatctggccggcgtcaaagaagtgatgatgccggttccggccttcaacgtcattaatggcggtagccatgcaggtaataaactggctatgcaggaatttatgattctgccgaccggtgccccgtcattcaccgaagccatgcgcatgggttcggaaatttatcatcacctgaaagcgctgattaagaaaaaatacggcctggatgcaacggctgttggtgacgaaggcggttttgccccgaacttccaagcgaatggcgaagccattgatctgctggttggtgcaatcgaaaaagctggctacaccggtaaaattaaaatcggcatggatgtcgcggcctccgaattctacaaaaacggtaaatacgatctggacttcaaaaatgaagaaagtaaagaagcggattggctgaccagcgaagccctgggcgaaatgtacaaaggtttcatcaaagatgccccggtgattagcatcgaagatccgtacgaccaggatgactgggaaggctggaccgcactgacgtctcagaccgatattcaaatcgtgggtgatgacctgaccgttacgaacccgaaacgtatccagatggcggttgataaaaaatcttgcaactgtctgctgctgaaagtcaatcaaattggctcagtgaccgaatcgatccgtgcgcataacctggccaaatctaatggctggggtacgatggtgtctcaccgctccggcgaaaccgaagattgcttcattgcagacctggtggttggcctgtgtacgggtcagatcaaaaccggtgctccgtgccgtagcgaacgcctgtctaaatataatcaactgctgcgcatcgaagaagaactgggtagcaatgcgaaatatgtgggtgataaattccgtatgccgttt

SEQ ID NO: 23 - DNA, 1134 bp, Artificial Sequence
  FBP aldolase expression DNA sequence
atgcatcatcaccatcaccacgaaaacctgtattttcagggcatgggtctggaaggcatcgttccgccgggtgtcattacgggtgataacctgattaaactgttcgaatactgccgcgaccacaaagtggcactgccggcttttaactgcaccagctctagtacgattaatgcagtgctgcaggcggcccgtgatattaaatctccggttatcgtccaatttagtaacggcggtgcagctttcatggcgggcaaaggtattaaaaatgatggccagaaagcctccgttctgggcgccatcgcaggtgctcaacatgttcgcctgatggccaaacactatggtgtcccggtggttctgcattctgatcactgcgcgaaaaaactgctgccgtggttcgatggcatgctggaagccgacgaagaatactttaaacagaacggtgaaccgctgttctcctcacacatgctggatctgtcggaagaatttgacgaagaaaatatcagcacctgtgcgaaatatttcacccgtatgacgaaaatgaaaatgtggctggaaatggaaattggcatcacgggcggtgaagaagatggtgtcgacaacaccaatgtgaaagccgaaagcctgtatacgaaaccggaacaggtctataacgtgtacaaaaccctgtccgaaattggcccgatgttttcaatcgcggccgcattcggcaacgttcatggtgtctataaagccggtaatgtcgtgctgtctccgcatctgctggctgatcaccagaaatacatcaaagaacaaatcaacagtccgctggacaaaccggcgtttctggtgatgcatggcggttcgggtagcacccgtgaagaaattgcggaagccgtgagcaacggtgttattaaaatgaatatcgataccgacacgcagtgggcatattgggatggcctgcgcaaattctacgaagaaaagaaagaatacctgcagggccaagttggtaacccggaaggtgctgataaaccgaataaaaaattctatgacccgcgtgtgtgggttcgtgctgccgaagaaagtatgatcaaacgcgctaacgaatcctttgaatccctgaacgcagtgaatgtgctgggtgacagttggaaacac

SEQ ID NO: 24 - DNA, 639 bp, Artificial Sequence
  PRX-2 expression DNA sequence
atgcaccatcaccaccaccacgaaaatctgtacttccaaggcatgtcactgcaaccgacgaacgacgccccgcaattcaaagcaatggcagtggttaacaaagaattcaaagaagtttcgctgaaagattacaccggcaaatacgtcgtgctgtttttctatccgctggactttaccttcgtctgcccgacggaaattatcgcatttggcgatcgtgcggccgacttccgcaaaattggttgcgaagtgctggcttgtagcaccgattctcatttcagtcatctgcactggatcaacacgccgcgtaaagaaggcggtctgggcgatatggacattccgctgatcgcagataaaaatatggaaatttcccgcgcttatggtgtcctgaaagaagatgacggcgtgtcatttcgtggtctgttcattatcgacggcacccagaaactgcgccaaattacgatcaatgatctgccggttggtcgttgcgtcgacgaaaccctgcgcctggttcaggcgtttcaatacacggatgtgcacggtgaagtttgtccggccggctggaaaccgggtaaaaaatctatgaaaccgtcaaaagaaggcgtgtcgtcctacctggcagatgctgaacaatccaaaaaa

SEQ ID NO: 25 - DNA, 788 bp, Artificial Sequence
  TIM expression DNA sequence
atgcatcatcatcatcatcacgaaaatctgtactttcaaggcatgggcggcggtcgcaaattctttgtcggcggcaactggaaaatgaacggcgataaaaaatctatcgatggtatcgtggattttctgagcaaaggcgatctggatccgaattgcgaagtggttgtgggtgcgagcccgtgttatctggattacagccgttctaaactgccggcaaacattggtgtggccgcacagaattgctataaagttgcgaaaggcgccttcaccggtgaaattagcccgcagatgatcaaagatgttggctgtgaatgggcaattctgggtcattctgaacgtcgcaacgtgtttggcgaaagtgatgaactgatcggtgaaaaagttgcattcgcgctggaaagcggcctgaaaattatcccgtgcatcggtgaaaaactggatgaacgcgaatctggtaaaacggaagaagtgtgttttaaacagctgaaagccatttctgataaagttagtgattgggatctggttgtgctggcgtatgaccggtgtgggcgattggtaccggtaaaaccgcaacgccggcacaggcacaggaaacccacctggcactgcgtaaatggctgaaagaaaacgttagcgaagaagtgtctcagaaagttcgcattctgtacggcggtagtgttagcgcgggcaattgcaaagaactgggtacccagccggatatcgatggcttcctggtgggtggtgcttccctgaaaccggactttgtgcagattatcaacgctacgaaa

SEQ ID NO: 26 - DNA, 558 bp, Artificial Sequence
  TCTP expression DNA sequence
atgcaccaccaccatcaccacgaaaatctgtacttccaaggcatgaaaatcttcaaagacgtgtttagcggcgacgaactgttctcggatacctacaaatttaaactgctggatgattgcctgtatgaagtgtacggcaaatatgttacccgtacggaaggcgatgtggttctggatggtgcgaacgccagcgcagaagaagcgatggatgattgtgatagctctagtacctctggtgtggatgtggttctgaatcatcgcctggttgaaaccggctttggtagcaagaaagattacacggtgtatctgaaagattacatgaagaaagtggttacgtatctggaagaaaacggcaaacaggcggaagtggataccttcaaaacgaacatcaacaaagttatgaaagaactgctgccgcgttttaaagatctgcagttctacaccggtgaaacgatggatccggaagccatgattatcatgctggaatataaagaagttgatggcaaagacattccggtgctgtacttcttcaaacacggcctgaacgaagaaaaattc

SEQ ID NO: 27 - protein, 446 aa, Artificial Sequence
  Antigen
MHHHHHHENLYFQGMPIKHIHARQIYDSRGNPTVEVDLTTERGIFRAAVPSGASTGVHEALELRDKDSTWHGKSGLKAVKNVNDVLGPELVKKNLDPVKQEEIDDFMISLDGTDNKSKFGANSILGISMAVCKAGAAHKGVPLYRHIADLAGVKEVMMPVPAFNVINGGSHAGNKLAMQEFMILPTGAPSFTEAMRMGSEIYHHLKALIKKKYGLDATAVGDEGGFAPNFQANGEAIDLLVGAIEKAGYTGKIKIGMDVAASEFYKNGKYDLDFKNEESKEADWLTSEALGEMYKGFIKDAPVISIEDPYDQDDWEGWTALTSQTDIQIVGDDLTVTNPKRIQMAVDKKSCNCLLLKVNQIGSVTESIRAHNLAKSNGWGTMVSHRSGETEDCFIADLVVGLCTGQIKTGAPCRSERLSKYNQLLRIEEELGSNAKYVGDKFRMPF

SEQ ID NO: 28 - protein, 378 aa, Artificial Sequence
  Antigen
MHHHHHHENLYFQGMGLEGIVPPGVITGDNLIKLFEYCRDHKVALPAFNCTSSSTINAVLQAARDIKSPVIVQFSNGGAAFMAGKGIKNDGQKASVLGAIAGAQHVRLMAKHYGVPVVLHSDHCAKKLLPWFDGMLEADEEYFKQNGEPLFSSHMLDLSEEFDEENISTCAKYFTRMTKMKMWLEMEIGITGGEEDGVDNTNVKAESLYTKPEQVYNVYKTLSEIGPMFSIAAAFGNVHGVYKAGNVVLSPHLLADHQKYIKEQINSPLDKPAFLVMHGGSGSTREEIAEAVSNGVIKMNIDTDTQWAYWDGLRKFYEEKKEYLQGQVGNPEGADKPNKKFYDPRVWVRAAEESMIKRANESFESLNAVNVLGDSWKH

SEQ ID NO: 29 - protein, 213 aa, Artificial Sequence
  Antigen
MHHHHHHENLYFQGMSLQPTNDAPQFKAMAVVNKEFKEVSLKDYTGKYVVLFFYPLDFTFVCPTEIIAFGDRAADFRKIGCEVLACSTDSHFSHLHWINTPRKEGGLGDMDIPLIADKNMEISRAYGVLKEDDGVSFRGLFIIDGTQKLRQITINDLPVGRCVDETLRLVQAFQYTDVHGEVCPAGWKPGKKSMKPSKEGVSSYLADAEQSKK

SEQ ID NO: 30 - protein, 263 aa, Artificial Sequence
  Antigen
MHHHHHHENLYFQGMGGGRKFFVGGNWKMNGDKKSIDGIVDFLSKGDLDPNCEVVVGASPCYLDYSRSKLPANIGVAAQNCYKVAKGAFTGEISPQMIKDVGCEWAILGHSERRNVFGESDELIGEKVAFALESGLKIIPCIGEKLDERESGKTEEVCFKQLKAISDKVSDWDLVVLAYEPVWAIGTGKTATPAQAQETHLALRKWLKENVSEEVSQKVRILYGGSVSAGNCKELGTQPDIDGFLVGGASLKPDFVQIINATK

SEQ ID NO: 31 - protein, 186 aa, Artificial Sequence
  Antigen
MHHHHHHENLYFQGMKIFKDVFSGDELFSDTYKFKLLDDCLYEVYGKYVTRTEGDVVLDGANASAEEAMDDCDSSSTSGVDVVLNHRLVETGFGSKKDYTVYLKDYMKKVVTYLEENGKQAEVDTFKTNINKVMKELLPRFKDLQFYTGETMDPEAMIIMLEYKEVDGKDIPVLYFFKHGLNEEKF

SEQ ID NO: 32 - RNA, 756 nt, Artificial Sequence
  mRNA for mCherry recombinant protein
atgcatcatcaccatcaccacgaaaacctgtattttcagggcatggtttccaaaggcgaagaagacaatatggcaatcatcaaagaatttatgcgtttcaaagtccacatggaaggttcagtcaatggccatgaatttgaaattgaaggcgaaggtgaaggccgtccgtatgaaggtacccagacggcaaaactgaaagtcaccaaaggcggtccgctgccgtttgcttgggatattctgtcaccgcaattcatgtatggttcgaaagcgtacgttaaacacccggccgatatcccggactacctgaaactgagctttccggaaggcttcaaatgggaacgtgttatgaacttcgaagatggcggtgtggttaccgtcacgcaggatagctctctgcaagacggtgaattcatctacaaagtgaaactgcgcggtaccaatttcccgtctgatggcccggttatgcagaagaaaaccatgggctgggaagcgagttccgaacgtatgtacccggaagacggtgccctgaaaggcgaaatcaaacagcgcctgaaactgaaagatggcggtcattatgacgcagaagtgaaaaccacgtacaaagctaaaaaaccggtccaactgccgggcgcatacaacgtgaacatcaaactggatatcaccagccacaacgaagactacacgatcgttgaacaatatgaacgtgcggaaggtcgtcactctacgggcggtatggatgaactgtacaaataatga

SEQ ID NO: 33 - protein, 250 aa, Artificial Sequence
  mCherry recombinant protein
MHHHHHHENLYFQGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 34 - DNA, 43 bp, Artificial Sequence
  Enolase 5′ RACE primer
gattacgccaagcttagtgcagagaccaacgacgagatcagcg

SEQ ID NO: 35 - DNA, 43 bp, Artificial Sequence
  Enolase 3′ RACE primer
gattacgccaagcttgccgttggagatgagggtggctttgctc

SEQ ID NO: 36 - DNA, 38 bp, Artificial Sequence
  FBP 5′ RACE primer
gattacgccaagcttcagcgccttctggatttccaacc

SEQ ID NO: 37 - DNA, 43 bp, Artificial Sequence
  FBP 3′ RACE primer
gattacgccaagcttggctccaccagagaagaaattgctgaag

SEQ ID NO: 38 - DNA, 43 bp, Artificial Sequence
  Prx-2 5′ RACE primer
gattacgccaagcttacactccatcgtcttccttgagcacgcc

SEQ ID NO: 39 - DNA, 43 bp, Artificial Sequence
  Prx-2 3′ RACE primer
gattacgccaagcttggtccttgcctgctccactgactcccat

SEQ ID NO: 40 - DNA, 43 bp, Artificial Sequence
  TIM 5′ RACE primer
gattacgccaagcttacgctctgaatgaccaagaatcgcccat

SEQ ID NO: 41 - DNA, 42 bp, Artificial Sequence
  TIM 3′ RACE primer
gattacgccaagcttgaggttgttgttggagcctcaccctgc

SEQ ID NO: 42 - DNA, 42 bp, Artificial Sequence
  TCTP 5′ RACE primer
gattacgccaagcttggaaccgaacccagtttcgaccagacg

SEQ ID NO: 43 - DNA, 43 bp, Artificial Sequence
  TCTP 3′ RACE primer
gattacgccaagcttctgctgaagaggccatggatgactgtga

SEQ ID NO: 44 - DNA, 43 bp, Artificial Sequence
  Enolase forward amplification primer
tatcgcctgcagaaaatgcctattaaacacattcatgcacgtc

SEQ ID NO: 45 - DNA, 38 bp, Artificial Sequence
  Enolase reverse amplification primer
atcgtagcggccgcttaaaagggcattctgaacttgtc

SEQ ID NO: 46 - DNA, 37 bp, Artificial Sequence
  FBP forward amplification primer
gctatcaagcttaaaatgggtcttgaaggaattgttc

SEQ ID NO: 47 - DNA, 34 bp, Artificial Sequence
  FBP reverse amplification primer
tcagatggatccttagtgtttccaggagtcacca

SEQ ID NO: 48 - DNA, 37 bp, Artificial Sequence
  Prx-2 forward amplification primer
tcgacgaagcttaaaatgagtcttcaaccaacgaatg

SEQ ID NO: 49 - DNA, 42 bp, Artificial Sequence
  Prx-2 reverse amplification primer
tcgactggtaccttatttctttgattgttcagcatctgcgag

SEQ ID NO: 50 - DNA, 36 bp, Artificial Sequence
  TIM forward amplification primer
tagctgggtaccttacttagtagcgttgatgatttg

SEQ ID NO: 51 - DNA, 39 bp, Artificial Sequence
  TIM reverse amplification primer
cgtatcaagcttaaaatgggtggaggaagaaaatttttc

SEQ ID NO: 52 - DNA, 37 bp, Artificial Sequence
  TCTP forward amplification primer
gtcattctgcagaaaatgaagatctttaaggacgtat

SEQ ID NO: 53 - DNA, 41 bp, Artificial Sequence
  TCTP reverse amplification primer
tcagtagcggccgcttaaaatttttcttcatttaatccatg

SEQ ID NO: 54 - DNA, 20 bp, Artificial Sequence
  pVAX1 forward sequencing primer - T7
taatacgactcactataggg

SEQ ID NO: 55 - DNA, 18 bp, Artificial Sequence
  pVAX1 reverse sequencing primer - BGH
tagaaggcacagtcgagg

SEQ ID NO: 56 - DNA, 25 bp, Artificial Sequence
  pVAC1 forward sequencing primer
acttggtgggtggagactgaagagt

SEQ ID NO: 57 - DNA, 23 bp, Artificial Sequence
  pVAC1 reverse sequencing primer
aggcaccacagaccttccaggat

SEQ ID NO: 58 - protein, 6 aa, Artificial Sequence
  6His tag
HHHHHH

SEQ ID NO: 59 - protein, 7 aa, Artificial Sequence
  TEV cleavage site
ENLYFQG

(19) United States
(12) Patent Application Publication
(10) Pub. No.: US 2021/0061866 A1
(43) Pub. Date: Mar. 4, 2021

(54) SEA LICE ANTIGENS AND VACCINES

(71) Applicants: BENCHMARK ANIMAL HEALTH LIMITED, Sheffield (GB); UNIVERSITY OF MAINE SYSTEM BOARD OF TRUSTEES, Orono, ME (US)

(72) Inventors: Sarah Elizabeth BARKER, Edinburgh (GB); Jessica Lynn PIESZ, Old Town, ME (US); Ian Robert BRICKNELL, Hampden, ME (US)

(21) Appl. No.: 16/963,645

(22) PCT Filed: Jan. 25, 2019
(86) PCT No.: PCT/GB2019/050216

§ 371 Date: Jul. 21, 2020

Related U.S. Application Data
(60) Provisional application No. 62/621,702, filed on Jan. 25, 2018.

(51) Int. Cl. C07K 14/435; A61K 39/00; A61P 33/14

(52) U.S. Cl. CPC C07K 14/43563; A61K 39/0003; A61K 2039/53; A61K 2039/70; A61K 2039/552; A61P 33/14

(57) ABSTRACT

Isolated proteins from caligid copepods, polynucleotides encoding the same, and antigens and vaccines comprising the same, in particular for the treatment or prevention of caligid copepod infection in fish. Proteins are peroxiredoxin-2 (Prx-2), fructose bisphosphate aldolase (FBP); enolase, transitionally-controlled tumour protein homolog (TCTP) and triosephosphate isomerase (TIM).

The present invention relates to isolated proteins from caligid copepods, and polynucleotides encoding the same, and antigens and vaccines comprising the same, in particular for the treatment or prevention of caligid copepod infection in fish.

Parasitic copepods in the family Caligidae (caligid copepods) infect and cause disease in fish. Collectively, these species are referred to as sea lice. There are three major genera of sea lice: Pseudocaligus, Caligus and Lepeophtheirus. In the northern hemisphere, the salmon louse (Lepeophtheirus salmonis), is responsible for most disease outbreaks on farmed salmonids. This parasite is responsible for substantial indirect and direct losses in aquaculture.

All developmental stages of sea lice, which are attached to the host, feed on host mucus, skin and blood. The attachment and feeding activities of sea lice result in lesions that vary in their nature and severity depending upon: the species of sea lice, their abundance, the developmental stages present and the species of the host (Johnson et al., 2004). In the southern hemisphere, Caligus rogercresseyi, is the primary caligid affecting the salmon farming industry in Chile (Gonzalez and Carvajal, 2003).

Caligid copepods have direct life cycles consisting of two free-living planktonic Nauplius stages, one free-swimming infectious copepodid stage, four to six attached chalimus stages, one or two preadult stages, and one adult stage (Kabata, 1970). Each of these developmental stages is separated by a moult. Once the adult stage is reached, caligid copepods do not undergo additional moults. In the case of L. salmonis, eggs hatch into the free-swimming first nauplius stage, which is followed by a second nauplius stage, and then the infectious copepodid stage. Once the copepodid locates a suitable host fish, it continues its development through four chalimus stages, first and second preadult stages, and then a final adult stage (Schram, 1993). The moults are characterized by gradual changes as the animal grows and undertakes physical modifications that enable it to live as a free-roaming parasite, feeding and breeding on the surface of the fish.

Feeding of caligid copepods on the mucus, skin and blood of their hosts leads to lesions that vary in severity based on the developmental stage(s) of the copepods present, the number of copepods present, their site(s) of attachment and the species of host. In situations of severe disease, such as is seen in Atlantic salmon (Salmo salar) when infected by high numbers of L. salmonis, extensive areas of skin erosion and haemorrhaging on the head and back, and a distinct area of erosion and sub-epidermal haemorrhage in the perianal region can be seen (Grimnes et al., 1996). Sea lice can cause physiological changes in their hosts including the development of a stress response, reduced immune function, osmoregulatory failure and death if untreated.

There are several management strategies that have been used for reducing the intensity of caligid copepod (sea lice) infestations. These include: fallowing of sites prior to restocking, year class separation and selection of farm sites to avoid areas where there are high densities of wild hosts or other environmental conditions suitable for sea lice establishment (Pike et al., 1999). Although the use of these strategies can in some cases lessen sea lice infection rates, their use individually or in combination has not been effective in eliminating infection.

A variety of chemicals and drugs have been used to control sea lice. These chemicals were designed for the control of terrestrial pests and parasites of plants and domestic animals. They include compounds such as hydrogen peroxide, organophosphates (e.g., dichlorvos and azamethiphos), ivermectin (and related compounds such as emamectin benzoate), insect molting regulators and pyrethrins (MacKinnon, 1997; Stone et al., 1999). Chemicals used in treatments are not necessarily effective against all of the stages of sea lice found on fish, and can create environmental risk. As seen in terrestrial pest and parasites there is evidence for the development of resistance in L. salmonis to some chemical treatments, especially in frequently-treated populations (Denholm, 2002). To reduce the costs associated with sea lice treatments and to eliminate environmental risks associated with these treatments new methods of sea lice control such as vaccines are needed.

A characteristic feature of attachment and feeding sites of caligid copepods on many of their hosts is a lack of a host immune response (Johnson et al., 2004; Jones et al., 1990; Jónsdóttir et al., 1992). This lack of an immune response is similar to that reported for other arthropod parasites such as ticks on terrestrial animals. In those instances, suppression of the host immune response is due to the production of immunomodulatory substances by the parasite (Wikel et al., 1996). These substances are being investigated for use as vaccine antigens to control these parasites. Sea lice, such as L. salmonis, like other arthropod ectoparasites, produce biologically active substances at the site of attachment and feeding that limits the host immune response. As these substances have potential for use in a vaccine against sea lice we have identified a number of these substances from L. salmonis and have examined their effects of host immune function in vitro.

Secretory proteins produced by the sea lice may act as immunomodulatory agents or assist in the feeding activities on the host (Fast et al., J Parasitol 89: 7-13, 2003, 2004). Neutralization of these activities by host-derived antibodies may impair sea lice growth and survival on salmon.

Vaccines are generally safer than chemical treatments, both to the fish and to the environment. Vaccine development has been hindered by a lack of knowledge of the host-pathogen interactions between sea lice and their hosts. There is therefore a need for further or improved commercial vaccines against sea lice.

WO 2006/010265 relates to recombinant vaccines against caligid copepods (sea lice) based on antigens isolated from sea lice.

The circum-oral glands are putative exocrine glands related to the mouth parts of sea lice. Isolated proteins from circum-oral glands may provide a source of potential antigens for use in vaccines against caligid copepods.

The present invention aims to provide alternative or improved vaccines and/or antigens or the treatment or prevention of caligid copepod infection in fish.

Accordingly, the present invention provides one or more isolated circum-oral gland (COG) protein for use for use in the treatment or prevention of caligid copepod infection in fish.

In embodiments of the invention, the or each protein is selected from the group consisting of: fructose bisphosphate aldolase (FBP); triosephosphate isomerase (TIM); peroxiredoxin-2 (Prx-2); enolase; and transitionally-controlled tumour protein homolog.

Transitionally Controlled Tumor Protein Homolog (TCTP)

TCTP is a highly conserved protein, expressed in all eukaryotic organisms. The protein sequence places it close to a family of small chaperone proteins and is often designated as a stress-related protein because TCTP expression is up-regulated during stress (Bommer and Thiele, 2004; Gnanasekar et al., 2009). For instance, TCTP can prevent hydrogen peroxide induced cell death (Nagano-Ito et al., 2009; 2012). The protein also functions in several cellular processes, such as cell growth, cell cycle progression, malignant transformation, and apoptosis (Boomer and Thiele, 2004). TCTP is also believed to have an extracellular cytokine-like function whereby it modulates the secretion of cytokines from mast cells, basophils, eosinophils, and T and B-lymphocytes (Boomer and Thiele, 2004; Sun et al., 2008). Parasites actively secrete TCTP proteins during host infection as part of their immune evasion strategy (Meyvis et al., 2009; Gnanasekar et al., 2002). Parasitic TCTP proteins have been shown to cause infiltration of eosinophils and/or histamine release from basophils (Bommer and Thiele, 2004; Gnanasekar et al., 2002). When TCTPs from Brugia malayi (Brug, 1927), a human filarial parasite, were injected intra-peritoneally into mice, an influx of eosinophils into the peritoneal cavity was observed suggesting filarial TCTP may play a role in allergic inflammatory responses in the host (Gnanasekar et al., 2002). In addition, intracellular expression of TCTP was shown to protect B. malayi against oxidative stress (Gnanasekar and Ramaswamy, 2007). The TCTP homolog from the parasite Schistosoma mansoni (Sambon, 1907), a human blood fluke, was shown to bind a variety of denatured proteins and protected the parasite from the effects of thermal shock (Gnanasekar et al., 2009). Knockdown of TCTP in Caenorhabditis elegans (Maupas, 1900), a free living nematode, using RNA interference resulted in the reduction in the number of eggs laid in the F₀ and F₁ generations by 90% and 72%, respectively, indicating the important role TCTP plays in reproduction (Meyvis et al., 2009). Interestingly, a TCTP from Plasmodium was shown to protect the parasite from the anti-malarial drug, artemisinin. Increased expression of TCTP correlated with increased resistance to the drug (Walker et al., 2000). These results suggest that the parasitic form of TCTP may be involved in certain pathological processes in the host.

Peroxiredoxin-2 (Prx-2)

Peroxiredoxins are a family of peroxidase proteins that are highly conserved and ubiquitously found in all living organisms. Their main role is to protect organisms from oxidative damage that can result from the generation of reactive oxygen species. 2-Cys peroxiredoxin produced in Fasciola gigantica (Cobbold, 1855), a parasite of livestock, was shown to reduce hydrogen peroxide levels and provide protection from oxidative damage (Sangpairoj et al., 2014). Some other proposed cellular functions include differentiation, apoptosis, and proliferation. Protein characterization studies in the hard tick have shown that Prx is expressed in all life stages of the parasite (Tsuji, Kamio et al. 2001). Using immunohistochemistry, Tsuji et al. (2001) was able to show strong Prx reactivity in the salivary glands of Haemaphysalis longicomis (tick). A DNA nicking assay showed H. longicornis recombinant Prx inhibits oxidative nicking of plasmid DNA (Tsuji et al., 2001). When the larval secretory-excretory antigens glyceraldehyde 3-phosphate dehydrogenase (G3PDH), a glycolytic enzyme, and Prx of the human trematode parasite S. mansoni were administered subcutaneously with papain, an allergen that induces T-helper 2 mediated responses, worm burdens and worm egg load in the liver and small intestine of mice were reduced 60-78% (El Ridi et al., 2013). Peroxiredoxin-2 secreted by F. hepatica and S. mansoni has been found to activate alternatively activated macrophages and induce a Th2 driven inflammatory response leading to an increase in IL-4, IL-5, and IL-13 secretion from naïve T helper cells (Donnelly et al., 2008).

Enolase

Enolase is a key glycolytic enzyme found in the cytoplasm of prokaryotic and eukaryotic cells that catalyzes the conversion of D-2-phosphoglycerate to phosphoenolpyruvate (PEP) and water. It is highly conserved and one of the most abundantly expressed cytosolic proteins of organisms and requires magnesium ions (Mg²⁺) to be enzymatically active (Diaz-Ramos et al., 2012). There are three different isoforms of α, β and γ. Alpha enolase is found in almost all human tissues whereas β and γ are found in muscle and neuron and/or neuroendocrine tissues, respectively (Diaz-Ramos et al., 2012). During cellular growth α-enolase is significantly upregulated. It has been identified in hematopoietic cells such as T and B cells, neuronal cells, monocytes, and endothelial cells as a plasminogen receptor (Diaz-Ramos et al., 2012). Studies have also shown that α-enolase can act as a heat-shock protein and a hypoxic stress protein. It is often referred to as a “moonlighting protein” because it has multiple functions at different cellular sites (Diaz-Ramos et al., 2012; Pal-Bhowmick et al., 2007). Enolase has been shown to bind plasmin in other parasitic models and aid in the invasion and migration within host tissues through its fibronolytic activity.

Triose Phosphate Isomerase (TIM a.k.a TPI)

Triose phosphate isomerase is a glycolytic enzyme that catalyzes the interconversion of glyceraldehyde 3-phosphate and dihydroxyacetone phosphate. Furthermore, the interaction of TIM on the surface of parasites (e.g. with lamin and fibronectin) suggests it might be an important virulence factor (Pereira et al., 2007). For example, in S. aureus, TIM is displayed at the cell surface and acts as an adhesion molecule (Furuya et al. 2011). Its location outside the cell suggests it might be important in the adherence and invasion of host tissues. The mechanism(s) of protection are not yet fully understood, however, vaccination studies with a TIM DNA vaccine has proven to be protective against S. japonicum in a mouse model. Mice vaccinated with the TIM DNA vaccine observed worm and egg reduction rates of 30.2% and 52.9% compared to the control (Zhu et al., 2004).

Fructose Bisphosphate Aldolase (FBP)

Fructose bisphosphate aldolase is a highly conserved enzyme in the glycolytic pathway that catalyzes the reversible cleavage of fructose-1,6-bisphosphate to dihydroxyacetone phosphate and glyceraldehyde 3-phosphate. Its primary importance is energy metabolism for all living things, but it also has been shown to induce strong humoral and cell mediated immune responses in parasitic infection models (McCarthy, Wieseman et al. 2002; Saber, Diab et al.

2013). For example, mice vaccinated with Schistosoma mansoni FBP DNA vaccine observed a significant reduction in worm burden and intestinal egg counts (Saber et al., 2013). Immunolocalization studies have also shown FBP aldolase is most highly expressed in metabolically active tissues and at all developmental stages of the parasite, Onchocerca volvulus (McCarthy et al., 2002).

In embodiments of the invention, the protein comprises the amino acid sequence of one or more of the group consisting of: SEQ ID NO:1; SEQ ID NO:2; SEQ ID NO:3; SEQ ID NO:4; SEQ ID NO:5; SEQ ID NO:6; and homologues thereof.

In all aspects of the present invention, “homologues” are sequences having at least 60%, at least 70%, at least 80%, at least 90%, at least 95%, at least 98%, or at least 99% identity to the recited sequence.

SEQ ID NO: 1:
MPIKHIHARQIYDSRGNPTVEVDLTTERGIFRAAVPSGASTGVHEA
LELRDKDSTWHGKSGLKAVKNVNDVLGPELVKKNLDPVKQEEIDDF
MISLDGTDNKSKFGANSILGISMAVCKAGAAHKGVPLYRHIADLAG
VKEVMMPVPAFNVINGGSHAGNKLAMQEFMILPTGAPSFTEAMRMG
SEIYHHLKALIKKKYGLDATAVGDEGGFAPNFQANGEAIDLLVGAI
EKAGYTGKIKIGMDVAASEFYKNGKYDLDFKNEESKEADWLTSEAL
GEMYKGFIKDAPVISIEDPYDQDDWEGWTALTSQTDIQIVGDDLTV
TNPKRIQMAVDKKSCNCLLLKVNQIGSVTESIRAHNLAKSNGWGTM
VSHRSGETEDCFIADLVVGLCTGQIKTGAPCRSERLSKYNQLLRIE
EELGSNAKYVGDKFRMPF
SEQ ID NO: 2:
MMPVPAFNVINGGSHAGNKLAMQEFMILPTGAPSFTEAMRMGSEIY
HHLKALIKKKYGLDATAVGDEGGFAPNFQANGEAIDLLVGAIEKAG
YTGKIKIGMDVAASEFYKNGKYDLDFKNEESKEADWLTSEALGEMY
KGFIKDAPVISIEDPYDQDDWEGRTALTSQTDIQIVGDDLTVTNPK
RIQMAVDKKSCNCLLLKVNQIGSVTESIRAHNLAKSNGWGTMVSHR
SGETEDCFIADLVVGLCTGQIKTGAPCRSERLSKYNQLLRIEEELG
SNAKYVGDKFRMPF
SEQ ID NO: 3:
MGLEGIVPPGVITGDNLIKLFEYCRDHKVALPAFNCTSSSTINAVL
QAARDIKSPVIVQFSNGGAAFMAGKGIKNDGQKASVLGAIAGAQHV
RLMAKHYGVPVVLHSDHCAKKLLPWFDGMLEADEEYFKQNGEPLFS
SHMLDLSEEFDEENISTCAKYFTRMTKMKMWLEMEIGITGGEEDGV
DNTNVKAESLYTKPEQVYNVYKTLSEIGPMFSIAAAFGNVHGVYKA
GNVVLSPHLLADHQKYIKEQINSPLDKPAFLVMHGGSGSTREEIAE
AVSNGVIKMNIDTDTQWAYWDGLRKFYEEKKEYLQGQVGNPEGADK
PNKKFYDPRVWVRAAEESMIKRANESFESLNAVNVLGDSWKH
SEQ ID NO: 4:
MSLQPTNDAPQFKAMAVVNKEFKEVSLKDYTGKYVVLFFYPLDFTF
VCPTEIIAFGDRAADFRKIGCEVLACSTDSHFSHLHWINTPRKEGG
LGDMDIPLIADKNMEISRAYGVLKEDDGVSFRGLFIIDGTQKLRQI
TINDLPVGRCVDETLRLVQAFQYTDVHGEVCPAGWKPGKKSMKPSK
EGVSSYLADAEQSKK
SEQ ID NO: 5:
MGGGRKFFVGGNWKMNGDKKSIDGIVDFLSKGDLDPNCEVVVGASP
CYLDYSRSKLPANIGVAAQNCYKVAKGAFTGEISPQMIKDVGCEWA
ILGHSERRNVFGESDELIGEKVAFALESGLKIIPCIGEKLDERESG
KTEEVCFKQLKAISDKVSDWDLVVLAYEPVWAIGTGKTATPAQAQE
THLALRKWLKENVSEEVSQKVRILYGGSVSAGNCKELGTQPDIDGF
LVGGASLKPDFVQIINATK
SEQ ID NO: 6:
MKIFKDVFSGDELFSDTYKFKLLDDCLYEVYGKYVTRTEGDVVLDG
ANASAEEAMDDCDSSSTSGVDVVLNHRLVETGFGSKKDYTVYLKDY
MKKVVTYLEENGKQAEVDTFKTNINKVMKELLPRFKDLQFYTGETM
DPEAMIIMLEYKEVDGKDIPVLYFFKHGLNEEKF

In embodiments of the invention, the protein is a recombinant protein.

An aspect of the invention provides an antigen comprising one or more protein according to the invention.

An aspect of the invention provides a vaccine against caligid copepod infection in fish, the vaccine comprising an immunologically effective amount of one or more protein according to the invention, and a pharmaceutically-acceptable diluent or carrier, and optionally an adjuvant.

In embodiments of the invention, each of the one or more antigens is different from the other antigen or antigens in the vaccine.

In embodiments of the invention, the vaccine comprises five antigens, wherein one of the five antigens comprises FBP, one of the five antigens comprises TIM, one of the five antigens comprises Prx-2, one of the five antigens comprises enolase, and one of the five antigens comprises TCTP.

In embodiments of the invention, the vaccine comprises five antigens, wherein one of the five antigens comprises the amino acid sequence of SEQ ID NO:1 or SEQ ID NO:2 or homologues thereof, one of the five antigens comprises the amino acid sequence of SEQ ID NO:3 or homologues thereof, one of the five antigens comprises the amino acid sequence of SEQ ID NO:4 or homologues thereof, one of the five antigens comprises the amino acid sequence of SEQ ID NO:5 or homologues thereof, and one of the five antigens comprises the amino acid sequence of SEQ ID NO:6 or homologues thereof.

In embodiments of the invention, the caligid copepod is Lepeophtheirus salmonis or Caligus rogercresseyi.

In embodiments of the invention, the fish is a salmonid. In embodiments of the invention, the fish is a salmon or trout.

As aspect of the invention provides, the protein, antigen or vaccine according to the invention for use in the treatment or prevention of caligid copepod infection in fish.

In embodiments of the invention, the caligid copepod is Lepeophtheirus salmonis or Caligus rogercresseyi.

In embodiments of the invention, the fish is a salmonid. In embodiments of the invention, the fish is a salmon or trout.

An aspect of the invention provides a polynucleotide comprising DNA encoding a protein isolated from the circum-oral gland (COG) or the frontal gland complex (FGC) of a caligid copepod.

In embodiments of the invention, the caligid copepod is Lepeophtheirus salmonis or Caligus rogercresseyi.

In embodiments of the invention, the protein encoded by the polynucleotide is selected from the group consisting of: fructose bisphosphate aldolase (FBP); triosephosphate isomerase (TIM); peroxiredoxin-2 (Prx-2); enolase; and transitionally-controlled tumour protein homolog (TCTP).

In embodiments of the invention, the polynucleotide according to the invention comprises DNA encoding the amino acid sequence of one or more of the group consisting of: SEQ ID NO:1; SEQ ID NO:2; SEQ ID NO:3; SEQ ID NO:4; SEQ ID NO:5; SEQ ID NO:6; and homologues thereof.

In embodiments of the invention, the polynucleotide according to the invention comprises DNA comprising the nucleotide sequence of one or more of the group consisting of: SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; and homologues thereof.

SEQ ID NO: 7:
ATGCCTATTAAACACATTCATGCACGTCAAATCTACGACTCTCGTGGTAACCCTACAGTGGA
GGTGGATCTCACCACTGAGCGAGGGATTTTCCGCGCTGCCGTCCCCAGTGGAGCTTCCACAG
GGGTTCATGAGGCCCTGGAACTGCGCGACAAGGACTCTACCTGGCACGGGAAGAGTGGTCTC
AAGGCTGTGAAGAATGTGAACGACGTCCTTGGGCCCGAGTTGGTGAAGAAGAACCTTGACCC
CGTGAAGCAAGAGGAGATCGATGATTTCATGATCAGCCTCGACGGGACGGATAACAAGAGCA
AATTTGGGGCTAATTCTATTTTGGGAATCTCGATGGCTGTGTGCAAGGCTGGTGCCGCCCAC
AAGGGTGTTCCCCTCTACCGCCATATCGCTGACTTGGCGGGTGTGAAGGAAGTGATGATGCC
GGTGCCCGCATTTAATGTCATTAACGGAGGTTCTCATGCTGGAAATAAGTTGGCGATGCAAG
AATTCATGATCCTTCCAACTGGAGCTCCCTCCTTCACTGAAGCCATGAGGATGGGATCTGAA
ATCTATCACCATCTCAAGGCTCTTATCAAGAAGAAGTACGGGTTGGATGCTACAGCCGTTGG
AGATGAGGGTGGCTTTGCTCCCAACTTCCAAGCCAACGGCGAGGCTATCGACCTTCTTGTTG
GAGCCATTGAAAAGGCTGGATACACTGGAAAAATCAAGATCGGAATGGATGTTGCTGCTTCA
GAATTTTACAAAAATGGAAAGTACGATTTAGATTTCAAAAATGAAGAATCCAAAGAGGCCGA
TTGGCTAACTTCCGAGGCTCTTGGTGAAATGTACAAAGGATTCATCAAGGATGCACCTGTCA
TTTCCATTGAAGATCCCTACGATCAAGATGATTGGGAGGGATGGACTGCATTGACATCACAA
ACTGACATTCAGATTGTCGGAGATGATCTCACAGTCACAAACCCCAAGCGTATTCAAATG
GCTGTTGACAAGAAATCTTGCAACTGCCTCCTCTTGAAAGTAAATCAAATTGGTTCAGTAAC
TGAATCTATTCGGGCCCACAATCTTGCTAAGAGCAACGGCTGGGGTACCATGGTCTCTCATA
GATCTGGTGAGACAGAGGATTGTTTCATCGCTGATCTCGTCGTTGGTCTCTGCACTGGTCAA
ATCAAGACTGGAGCTCCTTGCAGATCCGAACGTTTGTCTAAATACAATCAATTGTTGCGTAT
TGAAGAGGAGTTGGGATCCAACGCTAAATATGTCGGTGACAAGTTCAGAATGCCCTTTTAA
SEQ ID NO: 8:
ATGCCGATTAAACACATCCATGCCCGCCAAATCTATGACTCCCGTGGTAACCCGACCGTTGA
AGTTGACCTGACCACCGAACGTGGCATTTTTCGTGCCGCGGTGCCGAGCGGTGCATCTACGG
GTGTTCATGAAGCTCTGGAACTGCGCGATAAAGACTCAACCTGGCACGGCAAATCGGGTCTG
AAAGCGGTCAAAAACGTGAATGATGTTCTGGGCCCGGAACTGGTGAAGAAAAACCTGGACCC
GGTCAAACAGGAAGAAATTGATGACTTTATGATCAGCCTGGATGGTACCGACAACAAATCTA
AATTCGGCGCAAATAGTATTCTGGGTATCTCCATGGCAGTCTGTAAAGCTGGCGCAGCTCAT
AAAGGTGTGCCGCTGTATCGTCACATTGCGGATCTGGCCGGCGTCAAAGAAGTGATGATGCC
GGTTCCGGCCTTCAACGTCATTAATGGCGGTAGCCATGCAGGTAATAAACTGGCTATGCAGG
AATTTATGATTCTGCCGACCGGTGCCCCGTCATTCACCGAAGCCATGCGCATGGGTTCGGAA
ATTTATCATCACCTGAAAGCGCTGATTAAGAAAAAATACGGCCTGGATGCAACGGCTGTTGG
TGACGAAGGCGGTTTTGCCCCGAACTTCCAAGCGAATGGCGAAGCCATTGATCTGCTGGTTG
GTGCAATCGAAAAAGCTGGCTACACCGGTAAAATTAAAATCGGCATGGATGTCGCGGCCTCC
GAATTCTACAAAAACGGTAAATACGATCTGGACTTCAAAAATGAAGAAAGTAAAGAAGCGGA
TTGGCTGACCAGCGAAGCCCTGGGCGAAATGTACAAAGGTTTCATCAAAGATGCCCCGGTGA
TTAGCATCGAAGATCCGTACGACCAGGATGACTGGGAAGGCTGGACCGCACTGACGTCTCAG
ACCGATATTCAAATCGTGGGTGATGACCTGACCGTTACGAACCCGAAACGTATCCAGATGGC
GGTTGATAAAAAATCTTGCAACTGTCTGCTGCTGAAAGTCAATCAAATTGGCTCAGTGACCG
AATCGATCCGTGCGCATAACCTGGCCAAATCTAATGGCTGGGGTACGATGGTGTCTCACCGC
TCCGGCGAAACCGAAGATTGCTTCATTGCAGACCTGGTGGTTGGCCTGTGTACGGGTCAGAT
CAAAACCGGTGCTCCGTGCCGTAGCGAACGCCTGTCTAAATATAATCAACTGCTGCGCATCG
AAGAAGAACTGGGTAGCAATGCGAAATATGTGGGTGATAAATTCCGTATGCCGTTT
SEQ ID NO: 9:
ATGGGTCTTGAAGGAATTGTTCCCCCTGGTGTCATCACTGGAGACAATCTTATTAAGTTGTT
CGAATACTGCAGAGACCATAAAGTTGCTCTCCCTGCTTTCAACTGCACGTCTTCTTCAACCA
TCAATGCAGTTTTGCAAGCAGCACGGGACATTAAATCCCCTGTGATTGTTCAATTTTCCAAT
GGTGGAGCTGCTTTTATGGCCGGCAAAGGCATCAAAAATGACGGTCAAAAGGCTAGTGTCCT
TGGTGCAATTGCTGGGGCTCAACATGTTCGTTTAATGGCAAAGCACTATGGTGTTCCTGTAG
TTCTTCACTCTGATCACTGTGCTAAAAAACTCCTCCCATGGTTTGATGGAATGCTTGAAGCT
GATGAAGAGTATTTCAAACAAAATGGTGAACCTCTTTTCTCCAGTCACATGCTTGATCTCTC
GGAGGAGTTTGATGAAGAAAATATTTCCACTTGTGCAAAATATTTTACTCGCATGACTAAAA
TGAAAATGTGGTTAGAAATGGAAATTGGAATCACTGGGGGCGAAGAGGATGGTGTTGACAAT
ACCAATGTGAAAGCGGAGTCTCTTTACACCAAACCCGAACAAGTTTACAACGTGTACAAAAC
ACTCAGCGAAATTGGACCAATGTTTTCCATTGCTGCCGCTTTTGGAAACGTACATGGTGTAT
ACAAGGCAGGTAACGTTGTTCTTTCCCCACATTTGTTGGCTGATCATCAAAAATACATCAAG
GAGCAAATTAACTCCCCACTTGATAAACCCGCCTTCCTTGTCATGCACGGAGGCTCCGGCTC
CACCAGAGAAGAAATTGCTGAAGCAGTAAGCAACGGTGTGATCAAAATGAATATTGATACGG
ATACTCAATGGGCTTACTGGGATGGTCTCAGAAAGTTTTATGAAGAAAAGAAGGAGTATCTT
CAAGGACAGGTTGGAAATCCAGAAGGCGCTGACAAGCCAAACAAAAAGTTTTACGATCCACG
AGTTTGGGTTCGTGCTGCTGAGGAGTCTATGATTAAGAGAGCCAATGAATCCTTTGAATCAT
TAAACGCTGTGAATGTCCTTGGTGACTCCTGGAAACACTAA
SEQ ID NO: 10:
ATGGGTCTGGAAGGCATCGTTCCGCCGGGTGTCATTACGGGTGATAACCTGATTAAACTGTT
CGAATACTGCCGCGACCACAAAGTGGCACTGCCGGCTTTTAACTGCACCAGCTCTAGTACGA
TTAATGCAGTGCTGCAGGCGGCCCGTGATATTAAATCTCCGGTTATCGTCCAATTTAGTAAC
GGCGGTGCAGCTTTCATGGCGGGCAAAGGTATTAAAAATGATGGCCAGAAAGCCTCCGTTCT
GGGCGCCATCGCAGGTGCTCAACATGTTCGCCTGATGGCCAAACACTATGGTGTCCCGGTGG
TTCTGCATTCTGATCACTGCGCGAAAAAACTGCTGCCGTGGTTCGATGGCATGCTGGAAGCC
GACGAAGAATACTTTAAACAGAACGGTGAACCGCTGTTCTCCTCACACATGCTGGATCTGTC
GGAAGAATTTGACGAAGAAAATATCAGCACCTGTGCGAAATATTTCACCCGTATGACGAAAA
TGAAAATGTGGCTGGAAATGGAAATTGGCATCACGGGCGGTGAAGAAGATGGTGTCGACAAC
ACCAATGTGAAAGCCGAAAGCCTGTATACGAAACCGGAACAGGTCTATAACGTGTACAAAAC
CCTGTCCGAAATTGGCCCGATGTTTTCAATCGCGGCCGCATTCGGCAACGTTCATGGTGTCT
ATAAAGCCGGTAATGTCGTGCTGTCTCCGCATCTGCTGGCTGATCACCAGAAATACATCAAA
GAACAAATCAACAGTCCGCTGGACAAACCGGCGTTTCTGGTGATGCATGGCGGTTCGGGTAG
CACCCGTGAAGAAATTGCGGAAGCCGTGAGCAACGGTGTTATTAAAATGAATATCGATACCG
ACACGCAGTGGGCATATTGGGATGGCCTGCGCAAATTCTACGAAGAAAAGAAAGAATACCTG
CAGGGCCAAGTTGGTAACCCGGAAGGTGCTGATAAACCGAATAAAAAATTCTATGACCCGCG
TGTGTGGGTTCGTGCTGCCGAAGAAAGTATGATCAAACGCGCTAACGAATCCTTTGAATCCC
TGAACGCAGTGAATGTGCTGGGTGACAGTTGGAAACAC
SEQ ID NO: 11:
ATGAGTCTTCAACCAACGAATGATGCTCCTCAATTCAAGGCTATGGCCGTTGTGAACAAGGA
ATTCAAGGAGGTGTCACTCAAGGACTATACCGGCAAATACGTGGTTCTCTTTTTCTACCCCT
TGGACTTTACCTTTGTTTGCCCCACAGAAATCATTGCCTTTGGAGATCGGGCTGCAGATTTC
CGTAAAATTGGATGTGAGGTCCTTGCCTGCTCCACTGACTCCCATTTTTCTCATCTCCACTG
GATCAACACTCCTCGTAAGGAGGGAGGACTTGGGGACATGGACATTCCCCTCATTGCGGATA
AGAACATGGAAATTTCTAGAGCCTATGGCGTGCTCAAGGAAGACGATGGAGTGTCCTTCAGA
GGACTTTTCATCATTGACGGCACTCAGAAACTCCGTCAAATCACCATCAATGATCTTCCTGT
CGGAAGATGCGTAGACGAAACCTTAAGACTTGTACAAGCCTTCCAATACACGGACGTGCATG
GCGAGGTTTGCCCTGCGGGATGGAAGCCAGGAAAGAAGTCTATGAAGCCCAGCAAGGAAGGT
GTCTCATCTTACCTCGCAGATGCTGAACAATCAAAGAAATAA
SEQ ID NO: 12:
ATGTCACTGCAACCGACGAACGACGCCCCGCAATTCAAAGCAATGGCAGTGGTTAACAAAGA
ATTCAAAGAAGTTTCGCTGAAAGATTACACCGGCAAATACGTCGTGCTGTTTTTCTATCCGC
TGGACTTTACCTTCGTCTGCCCGACGGAAATTATCGCATTTGGCGATCGTGCGGCCGACTTC
CGCAAAATTGGTTGCGAAGTGCTGGCTTGTAGCACCGATTCTCATTTCAGTCATCTGCACTG
GATCAACACGCCGCGTAAAGAAGGCGGTCTGGGCGATATGGACATTCCGCTGATCGCAGATA
AAAATATGGAAATTTCCCGCGCTTATGGTGTCCTGAAAGAAGATGACGGCGTGTCATTTCGT
GGTCTGTTCATTATCGACGGCACCCAGAAACTGCGCCAAATTACGATCAATGATCTGCCGGT
TGGTCGTTGCGTCGACGAAACCCTGCGCCTGGTTCAGGCGTTTCAATACACGGATGTGCACG
GTGAAGTTTGTCCGGCCGGCTGGAAACCGGGTAAAAAATCTATGAAACCGTCAAAAGAAGGC
GTGTCGTCCTACCTGGCAGATGCTGAACAATCCAAAAAA
SEQ ID NO: 13:
ATGGGTGGAGGAAGAAAATTTTTCGTTGGTGGAAACTGGAAAATGAATGGAGACAAGAAATC
TATTGATGGAATCGTAGATTTTTTGAGCAAGGGGGATTTGGACCCAAATTGTGAGGTTGTTG
TTGGAGCCTCACCCTGCTATTTGGACTATTCCCGTTCTAAACTTCCTGCCAATATCGGAGTG
GCTGCACAAAATTGTTATAAGGTGGCCAAAGGAGCATTTACCGGAGAAATCAGTCCTCAAAT
GATTAAAGATGTTGGTTGTGAATGGGCGATTCTTGGTCATTCAGAGCGTAGAAATGTCTTTG
GGGAATCTGATGAGCTCATTGGCGAAAAGGTTGCTTTTGCACTTGAGTCTGGTCTCAAAATT
ATTCCATGCATTGGAGAAAAATTAGACGAACGTGAATCTGGGAAGACTGAGGAGGTCTGCTT
TAAGCAACTTAAAGCCATTTCTGACAAAGTATCTGATTGGGATCTTGTCGTCTTAGCTTATG
AACCAGTTTGGGCCATTGGAACTGGCAAAACAGCTACACCTGCTCAGGCTCAAGAAACACAT
CTTGCTCTTCGTAAATGGCTAAAGGAGAACGTTTCTGAGGAAGTTTCACAAAAAGTGCGAAT
CCTCTATGGAGGTTCCGTGAGTGCTGGTAATTGCAAGGAACTTGGCACTCAGCCTGATATTG
ACGGCTTCCTTGTTGGAGGAGCCTCTCTCAAACCTGACTTTGTTCAAATCATCAACGCTACT
AAGTAA
SEQ ID NO: 14:
ATGGGCGGCGGTCGCAAATTCTTTGTCGGCGGCAACTGGAAAATGAACGGCGATAAAAAATC
TATCGATGGTATCGTGGATTTTCTGAGCAAAGGCGATCTGGATCCGAATTGCGAAGTGGTTG
TGGGTGCGAGCCCGTGTTATCTGGATTACAGCCGTTCTAAACTGCCGGCAAACATTGGTGTG
GCCGCACAGAATTGCTATAAAGTTGCGAAAGGCGCCTTCACCGGTGAAATTAGCCCGCAGAT
GATCAAAGATGTTGGCTGTGAATGGGCAATTCTGGGTCATTCTGAACGTCGCAACGTGTTTG
GCGAAAGTGATGAACTGATCGGTGAAAAAGTTGCATTCGCGCTGGAAAGCGGCCTGAAAATT
ATCCCGTGCATCGGTGAAAAACTGGATGAACGCGAATCTGGTAAAACGGAAGAAGTGTGTTT
TAAACAGCTGAAAGCCATTTCTGATAAAGTTAGTGATTGGGATCTGGTTGTGCTGGCGTATG
AACCGGTGTGGGCGATTGGTACCGGTAAAACCGCAACGCCGGCACAGGCACAGGAAACCCAC
CTGGCACTGCGTAAATGGCTGAAAGAAAACGTTAGCGAAGAAGTGTCTCAGAAAGTTCGCAT
TCTGTACGGCGGTAGTGTTAGCGCGGGCAATTGCAAAGAACTGGGTACCCAGCCGGATATCG
ATGGCTTCCTGGTGGGTGGTGCTTCCCTGAAACCGGACTTTGTGCAGATTATCAACGCTACG
AAA
SEQ ID NO: 15:
ATGAAGATCTTTAAGGACGTATTTTCTGGAGATGAATTATTTTCCGACACCTACAAGTTCAA
GTTGTTGGATGATTGCTTGTACGAGGTGTATGGAAAGTATGTCACACGGACTGAAGGAGATG
TGGTTCTTGATGGAGCCAACGCATCTGCTGAAGAGGCCATGGATGACTGTGATTCCTCTTCC
ACCTCTGGTGTCGATGTTGTCCTTAACCACCGTCTGGTCGAAACTGGGTTCGGTTCCAAGAA
GGACTACACCGTATACCTTAAGGACTACATGAAGAAGGTAGTGACATATTTAGAAGAAAATG
GCAAACAAGCCGAAGTAGATACCTTCAAGACCAACATCAACAAGGTCATGAAGGAACTTTTA
CCACGGTTTAAGGATCTTCAATTCTATACTGGAGAAACGATGGACCCTGAGGCCATGATCAT
CATGCTTGAATACAAGGAAGTTGATGGAAAGGATATTCCCGTCCTCTACTTTTTTAAACATG
GATTAAATGAAGAAAAATTTTAA
SEQ ID NO: 16:
ATGAAAATCTTCAAAGACGTGTTTAGCGGCGACGAACTGTTCTCGGATACCTACAAATTTAA
ACTGCTGGATGATTGCCTGTATGAAGTGTACGGCAAATATGTTACCCGTACGGAAGGCGATG
TGGTTCTGGATGGTGCGAACGCCAGCGCAGAAGAAGCGATGGATGATTGTGATAGCTCTAGT
ACCTCTGGTGTGGATGTGGTTCTGAATCATCGCCTGGTTGAAACCGGCTTTGGTAGCAAGAA
AGATTACACGGTGTATCTGAAAGATTACATGAAGAAAGTGGTTACGTATCTGGAAGAAAACG
GCAAACAGGCGGAAGTGGATACCTTCAAAACGAACATCAACAAAGTTATGAAAGAACTGCTG
CCGCGTTTTAAAGATCTGCAGTTCTACACCGGTGAAACGATGGATCCGGAAGCCATGATTAT
CATGCTGGAATATAAAGAAGTTGATGGCAAAGACATTCCGGTGCTGTACTTCTTCAAACACG
GCCTGAACGAAGAAAAATTC

In embodiments of the invention, the DNA is cDNA.

An aspect of the invention provides an antigen comprising the polynucleotide according to the invention.

An aspect of the invention provides a vaccine against caligid copepod infection in fish, the vaccine comprising an immunologically effective amount of one or more polynucleotides according to the invention, or one or more antigen according to the invention, a pharmaceutically-acceptable diluent or carrier, and optionally an adjuvant.

In an embodiment of the invention, the vaccine comprises an immunologically effective amount of a combination of two or more antigens, wherein each of the one or more antigens independently comprises the DNA sequence selected from the group consisting of: SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; and homologues thereof.

In an embodiment of the invention, the one or more antigens is different from the other antigen or antigens in the vaccine.

In an embodiment of the invention, the vaccine comprises five antigens, wherein one of the five antigens comprises the DNA sequence of SEQ ID NO:7 or SEQ ID NO:8 or homologues thereof, one of the five antigens comprises the DNA sequence of SEQ ID NO:9 or SEQ ID NO:10 or homologues thereof, one of the five antigens comprises the DNA sequence of SEQ ID NO:11 or SEQ ID NO:12 or homologues thereof, one of the five antigens comprises the DNA sequence of SEQ ID NO:13 or SEQ ID NO:14 or homologues thereof, and one of the five antigens comprises the DNA sequence of SEQ ID NO:15 or SEQ ID NO:16 or homologues thereof.

In an embodiment of the invention, the caligid copepod is Lepeophtheirus salmonis or Caligus rogercresseyi.

In an embodiment of the invention, the fish is a salmonid. In an embodiment of the invention, the fish is a salmon or trout.

An aspect of the invention provides, the polynucleotide, antigen or vaccine according to the invention for use in the treatment or prevention of caligid copepod infection in fish.

In an embodiment of the invention, the caligid copepod infection is a Lepeophtheirus salmonis or Caligus rogercresseyi infection.

In an embodiment of the invention, the fish is a salmonid. In an embodiment of the invention, the fish is a salmon or trout.

An aspect of the invention provides, a method of treatment or prevention of caligid copepod infection in fish, comprising administering a therapeutic amount of the protein, polynucleotide, antigen, or vaccine of any one previous claim, optionally with the co-administration of an adjuvant.

In an embodiment of the invention, the caligid copepod infection is a Lepeophtheirus salmonis or Caligus rogercresseyi infection.

In an embodiment of the invention, the fish is a salmonid. In an embodiment of the invention, the fish is a salmon or trout.

The skilled person will appreciate that the claimed invention includes in its scope for the purposes of determining infringement variants of the claimed features that achieve substantially the same result in substantially the same way as the invention.

The invention will now be described by way of example with reference to the drawings in which:

FIG. 1 shows ELISA results for Atlantic salmon serum antibody response to TIM antigen with DNA antigen prime and protein boost;

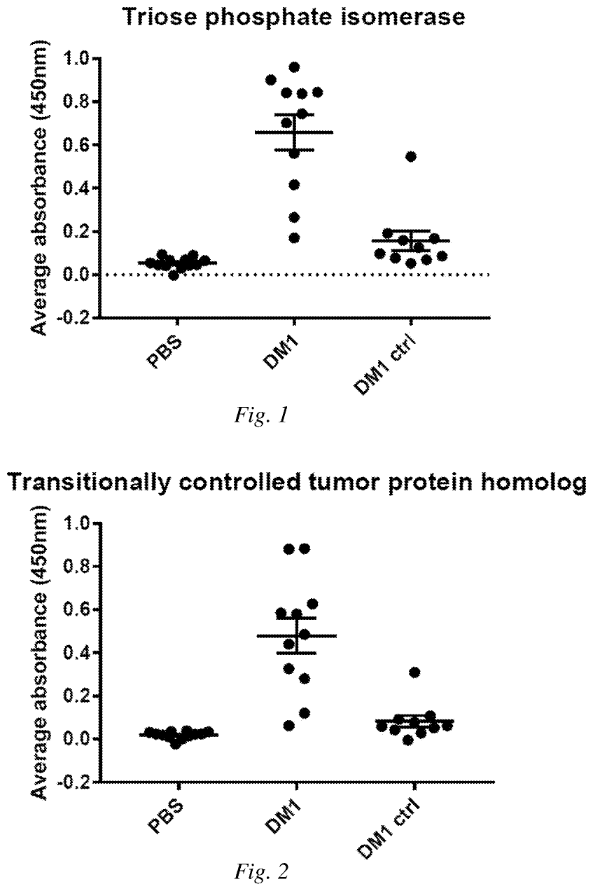

FIG. 2 shows ELISA results for Atlantic salmon serum antibody response to TCTP antigen with DNA antigen prime and protein boost;

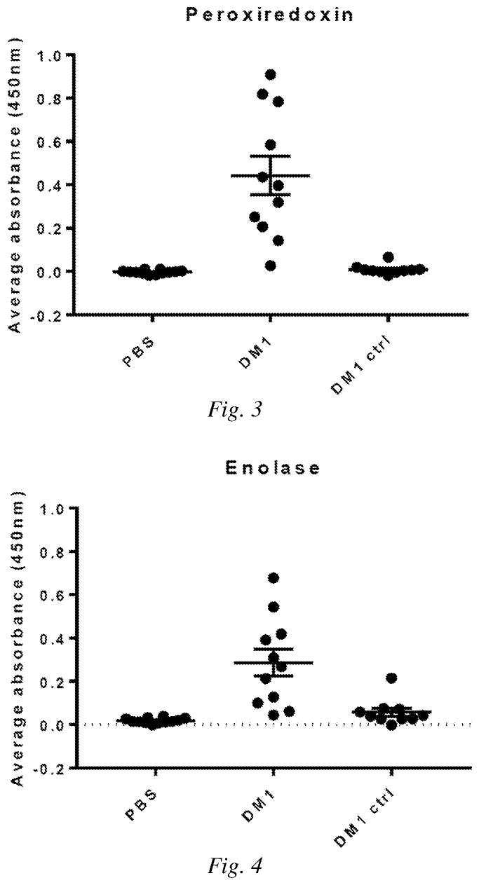

FIG. 3 shows ELISA results for Atlantic salmon serum antibody response to peroxiredoxin-2 antigen with DNA antigen prime and protein boost;

FIG. 4 shows ELISA results for Atlantic salmon serum antibody response to enolase antigen with DNA antigen prime and protein boost;

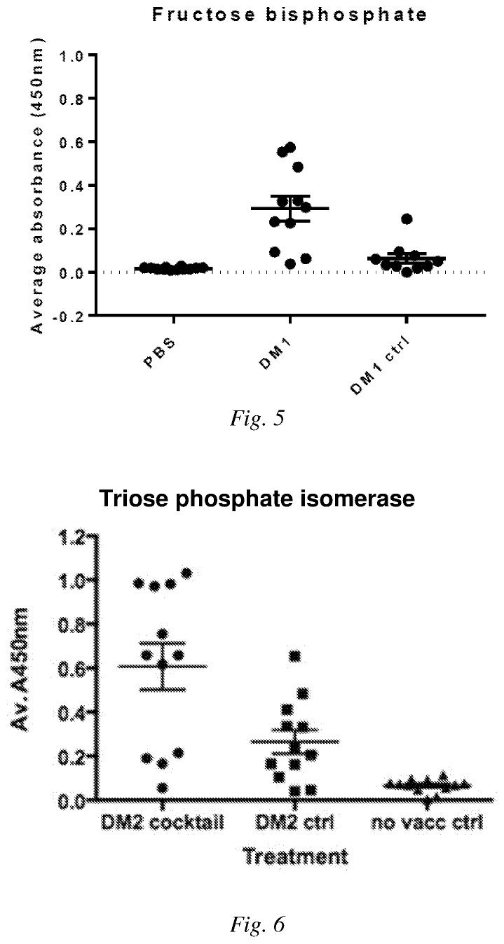

FIG. 5 shows ELISA results for Atlantic salmon serum antibody response to fructose bisphosphate antigen with DNA antigen prime and protein boost;

FIG. 6 shows ELISA results for Atlantic salmon serum antibody response to TIM antigen with protein antigen prime and protein boost;

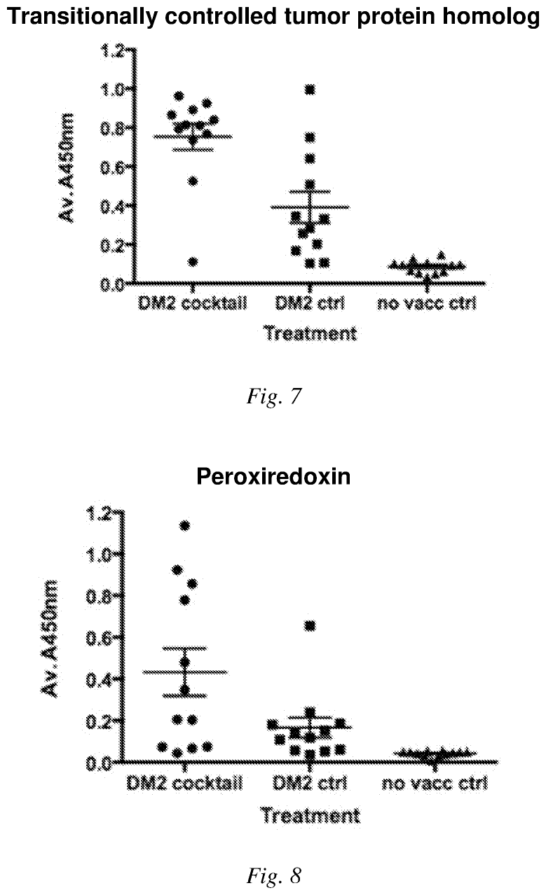

FIG. 7 shows ELISA results for Atlantic salmon serum antibody response to TCTP antigen with protein antigen prime and protein boost;

FIG. 8 shows ELISA results for Atlantic salmon serum antibody response to peroxiredoxin-2 antigen with protein antigen prime and protein boost;

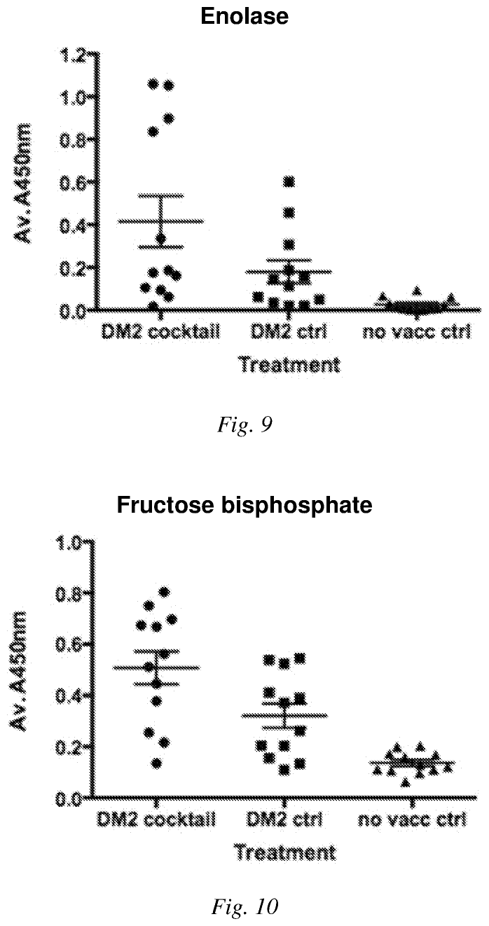

FIG. 9 shows ELISA results for Atlantic salmon serum antibody response to enolase antigen with protein antigen prime and protein boost; and

FIG. 10 shows ELISA results for Atlantic salmon serum antibody response to fructose bisphosphate antigen with protein antigen prime and protein boost.

For each of FIGS. 1 to 5, the data show the average absorbance at 450 nm. PBS is a vehicle control. DM1 (delivery method 1) is a vaccine prime using a cocktail of five DNA antigens (10 μg) with vaccine boost using cocktail of five recombinant proteins (50 μg). DM1 ctrl (delivery method 1 control) is a “prime” of DNA vaccine comprising empty pVAX1 vector (10 μg) with vaccine boost using mCherry recombinant protein (50 μg).

For each of FIGS. 6 to 10, the data show the blood serum IgM antibody response against the stated recombinant protein in individual Atlantic salmon parr vaccinated with the cocktail vaccine (DM2 cocktail), negative control fluorescent protein (DM2 ctrl) or no vaccination control (no vac ctrl) by delivery method 2 at 602 degree days post-vaccination at 14° C. Absorbance at 450 nm shown for individual fish (circles, squares or triangles) with line indicating mean±SEM (n=12 fish per group). DM2 cocktail (delivery method 2 cocktail) is a vaccine prime using a cocktail of five recombinant antigens (50 μg) with vaccine boost using cocktail of five recombinant proteins (50 μg). DM2 ctrl (delivery method 2 control) is a “prime” using mCherry-His recombinant protein (250 μg) plus flagellin (50 ng) with vaccine boost using mCherry-His recombinant protein (250 μg).

### EXAMPLES

### Example 1

### Isolation of Candidate Antigen Peptides from Circum-Oral Glands

The circum-oral glands (COGs) were visualized in L. salmonis at chalimus stages using 3,3′-diaminobenzidine tetrahydrochloride (DAB). COGs were isolated by microdissection and transferred into microcentrifuge tubes containing protease inhibitor cocktail (AEBSF [4-(2-aminoethyl) benzenesulfonyl fluoride] at 2 mM, Aprotinin at 0.3 μM, Bestatin at 116 μM, E-64 at 14 μM, Leupeptin at 1 μM and EDTA at 1 mM in 100 ml stock solution; Sigma-Aldrich Cat. No. P2714) at a 1 to 10 dilution in cold, sterile crustacean Ringers saline. Ringers saline was prepared by dissolving 0.58 M sodium chloride, 0.013 M potassium chloride, 0.013 M calcium chloride, 0.026 M magnesium chloride, 0.00054 M disodium hydrogen phosphate in 0.05M Tris-HCl, pH 7.5. Tissue was homogenised for two minutes at a frequency of 28 hertz using a TissueLyser II (Qiagen) by adding 100 μl 0.5 mm glass beads (BioSpec Products, catalog number 11079105) to 100 μl of sample. The supernatant was collected by centrifuging homogenate at 10,000×g for 30 minutes at 4° C. Protein concentration was determined using a BCA protein assay kit (Pierce Cat. No. 23227). The COG supernatant yielded 610 μg of protein. Samples were stored at −80° C.

Protein samples were concentrated with a 3K MWCO concentrator (Pierce) following manufacturer's instructions, and run on a SDS-PAGE gel. Gel slices containing proteins at 40 and 25 kDa were then analysed by nano-LC MS/MS.

Five proteins identified by the nano-LC MS/MS analysis were selected as candidate antigens: fructose bisphosphate aldolase (FBP; Hu et al., 2015; Lorenzatto et al., 2012); triosephosphate isomerase (TIM; Furuya et al. 2011; Saramago et al., 2012); peroxiredoxin-2 (Prx-2; Knoops et al., 2016; Rhee et al., 2016; Wood et al., 2003); enolase (Diaz-Ramos et al, 2012; Wang et al., 2013); and transitionally-controlled tumour protein homolog (TCTP; Gnanasekar et al., 2009; Gnanasekar and Ramaswamy, 2007; Sun et al., 2008; Nagano-Ito et al., 2009 and 2012).

### Example 2

### Production of Recombinant Vaccines from Circum-Oral Glands Peptides

The glycosylation of our protein targets was examined using NetNGlyc 1.0.

The server identified one potential N-linked glycosylation site for both FBP and TIM.

NetOGlyc 4.0 software identified two potential O-linked glycosylation sites for Prx-2.

Protein sequencing results from the nano-LC MS/MS analysis were used to blast NCBI database to obtain the complete mRNA coding sequence. As a quality control measure, the NCBI mRNA sequences of the targets were validated by performing RACE cDNA synthesis. To perform RACE cDNA synthesis, cDNA was prepared from RNA collected from 10 adult sea lice (RNeasyR Mini kit (Qiagen)). 5′ and 3′-RACE-Ready cDNA was prepared using a SMARTer RACE 5′/3′ cDNA synthesis kit (TaKaRa) for rapid amplification of cDNA ends.

Primers were specially designed for each protein to ensure amplification of the 5′ end (5′ RACE PCR) or 3′ end (3′ RACE PCR) of the mRNA (see Table 1 for list of primers used). PCR products were gel extracted using the NucleoSpin Gel and PCR clean up kit (Clontech).

TABLE
RACE primers

| Protein | 5′ RACE cDNA | 3′ RACE cDNA |
|---|---|---|
| FBP | GATTACGCCAAGCTTC | GATTACGCCAAGCTTGG |
| | AGCGCCTTCTGGATTT | CTCCACCAGAGAAGAAA |
| | CCAACC | TTGCTGAAG |
| | (SEQ ID NO: 36) | (SEQ ID NO: 37) |
| Enolase | GATTACGCCAAGCTTA | GATTACGCCAAGCTTGC |
| | GTGCAGAGACCAACGA | CGTTGGAGATGAGGGTG |
| | CGAGATCAGCG | GCTTTGCTC |
| | (SEQ ID NO: 34) | (SEQ ID NO: 35) |
| TIM | GATTACGCCAAGCTTA | GATTACGCCAAGCTTGA |
| | CGCTCTGAATGACCAA | GGTTGTTGTTGGAGCCT |
| | GAATCGCCCAT | CACCCTGC |
| | (SEQ ID NO: 40) | (SEQ ID NO: 41) |
| TCTP | GATTACGCCAAGCTTG | GATTACGCCAAGCTTCT |
| | GAACCGAACCCAGTTT | GCTGAAGAGGCCATGGA |
| | CGACCAGACG | TGACTGTGA |
| | (SEQ ID NO: 42) | (SEQ ID NO: 43) |
| Prx-2 | GATTACGCCAAGCTTA | GATTACGCCAAGCTTGG |
| | CACTCCATCGTCTTCC | TCCTTGCCTGCTCCACT |
| | TTGAGCACGCC | GACTCCCAT |
| | (SEQ ID NO: 38) | (SEQ ID NO: 39) |

In-Fusion cloning of RACE products was then performed following the manufacturer's instructions. Single colonies (8-10) were isolated from culture plates and grown overnight in selective media (ampicillin) at 37° C. with shaking (˜180 rpm). Plasmid DNA was isolated from bacterial lysates using a QIAprep Spin Miniprep Kit following the manufacturer's instructions (Qiagen). To determine which clones contained our RACE insert, we analyzed the DNA by restriction digest using EcoRI and HindIII which flank the cloning site. Digested products were visualized on a 1% ethidium bromide gel.

Clones containing the largest gene specific inserts were sequenced. The mRNA sequencing results are shown in below (coding region underlined):

Enolase mRNA (SEQ ID NO: 17):
GCTCCGATTCACTTCTTATTTCTCAACGCTCATCGATACTTTATAAGGCTCAAATTCAAAAT
GCCTATTAAACACATTCATGCACGTCAAATCTACGACTCTCGTGGTAACCCTACAGTGGAGG
TGGATCTCACCACTGAGCGAGGGATTTTCCGCGCTGCCGTCCCCAGTGGAGCTTCCACAGGG
GTTCATGAGGCCCTGGAACTGCGCGACAAGGACTCTACCTGGCACGGGAAGAGTGGTCTCAA
GGCTGTGAAGAATGTGAACGACGTCCTTGGGCCCGAGTTGGTGAAGAAGAACCTTGACCCCG
TGAAGCAAGAGGAGATCGATGATTTCATGATCAGCCTCGACGGGACGGATAACAAGAGCAAA
TTTGGGGCTAATTCTATTTTGGGAATCTCGATGGCTGTGTGCAAGGCTGGTGCCGCCCACAA
GGGTGTTCCCCTCTACCGCCATATCGCTGACTTGGCGGGTGTGAAGGAAGTGATGATGCCGG
TGCCCGCATTTAATGTCATTAACGGAGGTTCTCATGCTGGAAATAAGTTGGCGATGCAAGAA
TTCATGATCCTTCCAACTGGAGCTCCCTCCTTCACTGAAGCCATGAGGATGGGATCTGAAAT
CTATCACCATCTCAAGGCTCTTATCAAGAAGAAGTACGGGTTGGATGCTACAGCCGTTGGAG
ATGAGGGTGGCTTTGCTCCCAACTTCCAAGCCAACGGCGAGGCTATCGACCTTCTTGTTGGA
GCCATTGAAAAGGCTGGATACACTGGAAAAATCAAGATCGGAATGGATGTTGCTGCTTCAGA
ATTTTACAAAAATGGAAAGTACGATTTAGATTTCAAAAATGAAGAATCCAAAGAGGCCGATT
GGCTAACTTCCGAGGCTCTTGGTGAAATGTACAAAGGATTCATCAAGGATGCACCTGTCATT
TCCATTGAAGATCCCTACGATCAAGATGATTGGGAGGGATGGACTGCATTGACATCACAAAC
TGACATTCAGATTGTCGGAGATGATCTCACAGTCACAAACCCCAAGCGTATTCAAATGGCTG
TTGACAAGAAATCTTGCAACTGCCTCCTCTTGAAAGTAAATCAAATTGGTTCAGTAACTGAA
TCTATTCGGGCCCACAATCTTGCTAAGAGCAACGGCTGGGGTACCATGGTCTCTCATAGATC
TGGTGAGACAGAGGATTGTTTCATCGCTGATCTCGTCGTTGGTCTCTGCACTGGTCAAATCA
AGACTGGAGCTCCTTGCAGATCCGAACGTTTGTCTAAATACAATCAATTGTTGCGTATTGAA
GAGGAGTTGGGATCCAACGCTAAATATGTCGGTGACAAGTTCAGAATGCCCTTTTAATGATC
TAAAGGGTTGTTTCTTCATTGAAGAAAGTTCATTTCTATAGTCACAATAAATTATTTCATGG
TTTTACAAGAAATTCACAGGACGAAAAAACAAAAATCTTAATTTATTGAATTATTTCTATAT
GTATTACACGCGTACTCTAAGTAAAACCTTATAAAGGAATATAATTGTAATATAATTTATTG
TAATATTTTTTTTTTCATATTTAATTTATATTAAGGGTTGCCATTTAAATATATAAATTCCC
CGTTGGTAAAAAAAAAAA
FBP aldolase mRNA (SEQ ID NO: 18):
GGGGGGAGTTAGTATAAGAGATCGACAGGCTCTGTTCGCAACACTTGTTCCTAAAGGCAAAT
TATCTTAAATCTTAAAAATGGGTCTTGAAGGAATTGTTCCCCCTGGTGTCATCACTGGAGAC
AATCTTATTAAGTTGTTCGAATACTGTAGAGACCATAAAGTTGCTCTCCCTGCTTTCAACTG
CACGTCTTCTTCAACCATCAATGCAGTTTTGCAAGCAGCACGGGACATTAAATCCCCTGTGA
TTGTTCAATTTTCCAATGGTGGAGCTGCTTTTATGGCCGGCAAAGGCATCAAAAATGACGGT
CAAAAGGCTAGTGTCCTTGGTGCAATTGCTGGGGCTCAACATGTTCGTTTAATGGCAAAGCA
CTATGGTGTTCCTGTAGTTCTTCACTCTGATCACTGTGCTAAAAAACTCCTCCCATGGTTTG
ATGGAATGCTTGAAGCTGATGAAGAGTATTTCAAACAAAATGGTGAACCTCTTTTCTCCAGT
CACATGCTTGATCTCTCGGAGGAGTTTGATGAAGAAAATATTTCCACTTGTGCAAAATATTT
TACTCGCATGACTAAAATGAAAATGTGGTTAGAAATGGAAATTGGAATCACTGGGGGCGAAG
AGGATGGTGTTGACAATACCAATGTGAAAGCGGAGTCTCTTTACACCAAACCCGAACAAGTT
TACAACGTGTACAAAACACTCAGCGAAATTGGACCAATGTTTTCCATTGCTGCCGCTTTTGG
AAACGTACATGGTGTATACAAGGCAGGTAACGTTGTTCTTTCCCCACATTTGTTGGCTGATC
ATCAAAAATACATCAAGGAGCAAATTAACTCCCCACTTGATAAACCCGCCTTCCTTGTCATG
CACGGAGGCTCCGGCTCCACCAGAGAAGAAATTGCTGAAGCAGTAAGCAACGGTGTGATCAA
AATGAATATTGATACGGATACTCAATGGGCTTACTGGGATGGTCTCAGAAAGTTTTATGAAG
AAAAGAAGGAGTATCTTCAAGGACAGGTTGGAAATCCAGAAGGCGCTGACAAGCCAAACAAA
AAGTTTTACGATCCACGAGTTTGGGTTCGTGCTGCTGAGGAGTCTATGATTAAGAGAGCCAA
TGAATCCTTTGAATCATTAAACGCTGTGAATGTCCTTGGTGACTCCTGGAAACACTAAATAC
TTATTATTGGATATTCAGAATGTTTTAATTTCTATTTTGGAACTCCGAACTTACTAGTAATT
TATTTCTCTTTTAAAAAATGAATCAGTATATTTATTATTCTGTTTATAAAATTAAGTTATTG
TTAATTTCCTTAAATTTATTTATCAAAAATTAGAAATTGTTATACATGAAACATTGACATAA
ATCTAAAATTGAAACATTTTATGATTTTGATGTTTATAAATGCTAGATAAGAAGTCATAAAT
AAATGTATAATAAATTAAACTTCTTTCGTGATTAATTAACTTGCTAATTAATGCATAATTTT
CATTTTTTTGAAGATATGCGCTAAAAAATTATTCAATAAAAATTAAAATAG
PRX-2 mRNA (SEQ ID NO: 19):
GGGGGAGTCTTATATCTGCTACCGGCAAGTGAACTACCTCTGTCATCTCTCTTTGTAATATC
CGACTAAGTAACAAAATGAGTCTTCAACCAACGAATGATGCTCCTCAATTCAAGGCTATGGC
CGTTGTGAACAAGGAATTCAAGGAGGTGTCACTCAAGGACTATACCGGCAAATACGTGGTTC
TCTTTTTCTACCCCTTGGACTTTACCTTTGTTTGCCCCACAGAAATCATTGCCTTTGGAGAT
CGGGCTGCAGATTTCCGTAAAATTGGATGTGAGGTCCTTGCCTGCTCCACTGACTCCCATTT
TTCTCATCTCCACTGGATCAACACTCCTCGTAAGGAGGGAGGACTTGGGGACATGGACATTC
CCCTCATTGCGGATAAGAACATGGAAATTTCTAGAGCCTATGGCGTGCTCAAGGAAGACGAT
GGAGTGTCCTTCAGAGGACTTTTCATCATTGACGGCACTCAGAAACTCCGTCAAATCACAAT
CAATGATCTTCCTGTCGGAAGATGCGTAGACGAAACCTTAAGACTTGTACAAGCCTTCCAAT
ACACAGACGTGCATGGCGAGGTTTGCCCTGCGGGATGGAAGCCAGGAAAGAAGTCTATGAAG
CCCAGCAAGGAAGGTGTCTCATCTTACCTCGCAGATGCTGAACAATCAAAGAAATAATACAG
AAGATCTCCCCTGTAGTTATTAGTTTCCATACCAATTCTCTCTTTTAATTCATTCGATTGGA
CACTGTTACCATGTTCCACTTTTTAATTGTACCTGGTCAGTCAGTGCCCAAGGTCATTGATT
GATTAAGTCTATCAAATATTTATGTATTCCCCGGTGTACTAATAGTTTTTAAGATATAAAAT
ATACGACTTTTTAATATATT
TIM mRNA (SEQ ID NO: 20):
GGGGGAGTTATAAAGCACTACTCGATTGCTAAGTACTTCGCGAGGTTCCTACTAATTGTAAT
ATAGTTGAAAAAATACATTCAAAAATGGGTGGAGGAAGAAAATTTTTCGTTGGTGGAAACTG
GAAAATGAATGGAGACAAGAAATCTATTGATGGAATCGTAGATTTTTTGAGCAAGGGGGATT
TGGACCCAAATTGTGAGGTTGTTGTTGGAGCCTCACCCTGCTATTTGGACTATTCCCGTTCT
AAACTTCCTGCCAATATCGGAGTGGCTGCACAAAATTGTTATAAGGTGGCCAAAGGAGCATT
TACCGGAGAAATCAGTCCTCAAATGATTAAAGATGTTGGTTGTGAATGGGCGATTCTTGGTC
ATTCAGAGCGTAGAAATGTCTTTGGGGAATCTGATGAGCTCATTGGCGAAAAGGTTGCTTTT
GCACTTGAGTCTGGTCTCAAAATTATTCCATGCATTGGAGAAAAATTAGACGAACGTGAATC
TGGGAAGACTGAGGAGGTCTGCTTTAAGCAACTTAAAGCCATTTCTGACAAAGTATCTGATT
GGGATCTTGTCGTCTTAGCTTATGAACCAGTTTGGGCCATTGGAACTGGCAAAACAGCTACA
CCTGCTCAGGCTCAAGAAACACATCTTGCTCTTCGTAAATGGCTAAAGGAGAACGTTTCTGA
GGAAGTTTCACAAAAAGTGCGAATCCTCTATGGAGGTTCCGTGAGTGCTGGTAATTGCAAGG
AACTTGGCACTCAGCCTGATATTGACGGCTTCCTTGTTGGAGGAGCCTCTCTCAAACCTGAC
TTTGTTCAAATCATCAACGCTACTAAGTAAACAAAATACTGGATATTCGACTCTTCTATAAT
AGTCTTATCATCTCTTTAATGCTCTCACTCATTATTTGATAAATAACGAGGTTAAAATATTA
TTTATTTGATTAAACGTAATCTAACGTAATACATATATATTAATTTTCACGAATGCAGAAAA
AAAATTATTGCATAAATACGTATTTTACA
TCTP mRNA (SEQ ID NO: 21):
GAGGTTGTCGGCTTTCAAGGACCACTCAATTCCTCCCTAGTTCTAATTCACTTTCACTCCGG
ACTCTTCCCGTAAACACTCCTGCCTTATACAAAATGAAGATCTTTAAGGACGTATTTTCTGG
AGATGAATTATTTTCCGACACCTACAAGTTCAAGTTGTTGGATGATTGCTTGTACGAGGTGT
ATGGAAAGTATGTCACACGGACTGAAGGAGATGTGGTTCTTGATGGAGCCAACGCATCTGCT
GAAGAGGCCATGGATGACTGTGATTCCTCTTCCACCTCTGGTGTCGATGTTGTCCTTAACCA
CCGTCTGGTCGAAACTGGGTTCGGTTCCAAGAAGGACTACACCGTATACCTTAAGGACTACA
TGAAGAAGGTAGTGACATATTTAGAAGAAAATGGCAAACAAGCCGAAGTAGATACCTTCAAG
ACCAACATCAACAAGGTCATGAAGGAACTTTTACCACGGTTTAAGGATCTTCAATTCTATAC
TGGAGAAACGATGGACCCTGAGGCCATGATCATCATGCTTGAATACAAGGAAGTTGATGGAA
AGGATATTCCCGTCCTCTACTTTTTTAAACATGGATTAAATGAAGAAAAATTTTAAACATTA
GTGTCATCATTCATCTCAATTTCTTATAAATGTTTATATCTACAATATATTTTATATAGATA
AAAAAGAATTTCCGTTGACAATAATATGCGAACTACCTAATTAAATTATGTTGTATTCATAT
TTCTAATGCGATTTTTGGGAAATTTCTCGTTATAACTAAATTCCATTTTTAACGTACACGTC
TGTATATGAATATATGTAAAGTGTTATTTACTTGTAAGAC

The mRNA sequencing data of the target proteins was aligned and compared with the corresponding NCBI mRNA sequence using the Clustal Omega multiple sequence alignment tool (EMBL-EBI).

In most cases, mRNA sequence data matched exactly or very closely (only single base pair differences) to the NCBI database, however, for one protein, enolase, an additional isoform was identified (new start codon identified upstream from the start site of the NCBI sequence). Using UniProt (Universal Protein Resource) the sequence matched with 99% identity to Tribolium castaneum, the red flour beetle. Both the red flour beetle (hexapod) and sea louse (crustacean) belong to the clade Pancrustacea in the phylum arthropoda (www.uniprot.org).

The characteristics of sea lice antigens are provided in Table 2.

TABLE 2
Characteristics of sea lice antigens

| | Protein Size | Amino acid length | mRNA length |
|---|---|---|---|
| | (kDa) | (residues) | (bp) |
| FBP-aldolase | 42.1 | 364 | 1539 |
| Prx-2 | 24 | 199 | 888 |
| Enolase | 48.9 | 432 | 1630 |
| Enolase (short) | 31.8 | 290 | 1146 |
| TCTP | 21.6 | 172 | 846 |
| TIM | 28.7 | 249 | 1021 |

The edited sequences were used to produce the protein antigens by recombinant protein production in E. coli. The DNA sequence for each protein was codon optimized prior to gene synthesis and cloned into the pET-30a (+) expression vector with N-terminal His tag along with TEV cleavage site. Recombinant plasmids were then transformed into E. coli BL21 (DE3) cells and grown overnight at 37° C. A single colony was selected and inoculated into 1 litre of LB media containing kanamycin and incubated at 200 rpm at 37° C.

The expression DNA sequences were as set out below:

Enolase (SEQ ID NO: 22):
ATGCATCATCACCATCACCACGAAAACCTGTATTTTCAGGGCATGCCGATTAAACACATCCA
TGCCCGCCAAATCTATGACTCCCGTGGTAACCCGACCGTTGAAGTTGACCTGACCACCGAAC
GTGGCATTTTTCGTGCCGCGGTGCCGAGCGGTGCATCTACGGGTGTTCATGAAGCTCTGGAA
CTGCGCGATAAAGACTCAACCTGGCACGGCAAATCGGGTCTGAAAGCGGTCAAAAACGTGAA
TGATGTTCTGGGCCCGGAACTGGTGAAGAAAAACCTGGACCCGGTCAAACAGGAAGAAATTG
ATGACTTTATGATCAGCCTGGATGGTACCGACAACAAATCTAAATTCGGCGCAAATAGTATT
CTGGGTATCTCCATGGCAGTCTGTAAAGCTGGCGCAGCTCATAAAGGTGTGCCGCTGTATCG
TCACATTGCGGATCTGGCCGGCGTCAAAGAAGTGATGATGCCGGTTCCGGCCTTCAACGTCA
TTAATGGCGGTAGCCATGCAGGTAATAAACTGGCTATGCAGGAATTTATGATTCTGCCGACC
GGTGCCCCGTCATTCACCGAAGCCATGCGCATGGGTTCGGAAATTTATCATCACCTGAAAGC
GCTGATTAAGAAAAAATACGGCCTGGATGCAACGGCTGTTGGTGACGAAGGCGGTTTTGCCC
CGAACTTCCAAGCGAATGGCGAAGCCATTGATCTGCTGGTTGGTGCAATCGAAAAAGCTGGC
TACACCGGTAAAATTAAAATCGGCATGGATGTCGCGGCCTCCGAATTCTACAAAAACGGTAA
ATACGATCTGGACTTCAAAAATGAAGAAAGTAAAGAAGCGGATTGGCTGACCAGCGAAGCCC
TGGGCGAAATGTACAAAGGTTTCATCAAAGATGCCCCGGTGATTAGCATCGAAGATCCGTAC
GACCAGGATGACTGGGAAGGCTGGACCGCACTGACGTCTCAGACCGATATTCAAATCGTGGG
TGATGACCTGACCGTTACGAACCCGAAACGTATCCAGATGGCGGTTGATAAAAAATCTTGCA
ACTGTCTGCTGCTGAAAGTCAATCAAATTGGCTCAGTGACCGAATCGATCCGTGCGCATAAC
CTGGCCAAATCTAATGGCTGGGGTACGATGGTGTCTCACCGCTCCGGCGAAACCGAAGATTG
CTTCATTGCAGACCTGGTGGTTGGCCTGTGTACGGGTCAGATCAAAACCGGTGCTCCGTGCC
GTAGCGAACGCCTGTCTAAATATAATCAACTGCTGCGCATCGAAGAAGAACTGGGTAGCAAT
GCGAAATATGTGGGTGATAAATTCCGTATGCCGTTT
FBP aldolase (SEQ ID NO: 23):
ATGCATCATCACCATCACCACGAAAACCTGTATTTTCAGGGCATGGGTCTGGAAGGCATCGT
TCCGCCGGGTGTCATTACGGGTGATAACCTGATTAAACTGTTCGAATACTGCCGCGACCACA
AAGTGGCACTGCCGGCTTTTAACTGCACCAGCTCTAGTACGATTAATGCAGTGCTGCAGGCG
GCCCGTGATATTAAATCTCCGGTTATCGTCCAATTTAGTAACGGCGGTGCAGCTTTCATGGC
GGGCAAAGGTATTAAAAATGATGGCCAGAAAGCCTCCGTTCTGGGCGCCATCGCAGGTGCTC
AACATGTTCGCCTGATGGCCAAACACTATGGTGTCCCGGTGGTTCTGCATTCTGATCACTGC
GCGAAAAAACTGCTGCCGTGGTTCGATGGCATGCTGGAAGCCGACGAAGAATACTTTAAACA
GAACGGTGAACCGCTGTTCTCCTCACACATGCTGGATCTGTCGGAAGAATTTGACGAAGAAA
ATATCAGCACCTGTGCGAAATATTTCACCCGTATGACGAAAATGAAAATGTGGCTGGAAATG
GAAATTGGCATCACGGGCGGTGAAGAAGATGGTGTCGACAACACCAATGTGAAAGCCGAAAG
CCTGTATACGAAACCGGAACAGGTCTATAACGTGTACAAAACCCTGTCCGAAATTGGCCCGA
TGTTTTCAATCGCGGCCGCATTCGGCAACGTTCATGGTGTCTATAAAGCCGGTAATGTCGTG
CTGTCTCCGCATCTGCTGGCTGATCACCAGAAATACATCAAAGAACAAATCAACAGTCCGCT
GGACAAACCGGCGTTTCTGGTGATGCATGGCGGTTCGGGTAGCACCCGTGAAGAAATTGCGG
AAGCCGTGAGCAACGGTGTTATTAAAATGAATATCGATACCGACACGCAGTGGGCATATTGG
GATGGCCTGCGCAAATTCTACGAAGAAAAGAAAGAATACCTGCAGGGCCAAGTTGGTAACCC
GGAAGGTGCTGATAAACCGAATAAAAAATTCTATGACCCGCGTGTGTGGGTTCGTGCTGCCG
AAGAAAGTATGATCAAACGCGCTAACGAATCCTTTGAATCCCTGAACGCAGTGAATGTGCTG
GGTGACAGTTGGAAACAC
Prx-2 (SEQ ID NO: 24):
ATGCACCATCACCACCACCACGAAAATCTGTACTTCCAAGGCATGTCACTGCAACCGACGAA
CGACGCCCCGCAATTCAAAGCAATGGCAGTGGTTAACAAAGAATTCAAAGAAGTTTCGCTGA
AAGATTACACCGGCAAATACGTCGTGCTGTTTTTCTATCCGCTGGACTTTACCTTCGTCTGC
CCGACGGAAATTATCGCATTTGGCGATCGTGCGGCCGACTTCCGCAAAATTGGTTGCGAAGT
GCTGGCTTGTAGCACCGATTCTCATTTCAGTCATCTGCACTGGATCAACACGCCGCGTAAAG
AAGGCGGTCTGGGCGATATGGACATTCCGCTGATCGCAGATAAAAATATGGAAATTTCCCGC
GCTTATGGTGTCCTGAAAGAAGATGACGGCGTGTCATTTCGTGGTCTGTTCATTATCGACGG
CACCCAGAAACTGCGCCAAATTACGATCAATGATCTGCCGGTTGGTCGTTGCGTCGACGAAA
CCCTGCGCCTGGTTCAGGCGTTTCAATACACGGATGTGCACGGTGAAGTTTGTCCGGCCGGC
TGGAAACCGGGTAAAAAATCTATGAAACCGTCAAAAGAAGGCGTGTCGTCCTACCTGGCAGA
TGCTGAACAATCCAAAAAA
TIM (SEQ ID NO: 25):
ATGCATCATCATCATCATCACGAAAATCTGTACTTTCAAGGCATGGGCGGCGGTCGCAAATT
CTTTGTCGGCGGCAACTGGAAAATGAACGGCGATAAAAAATCTATCGATGGTATCGTGGATT
TTCTGAGCAAAGGCGATCTGGATCCGAATTGCGAAGTGGTTGTGGGTGCGAGCCCGTGTTAT
CTGGATTACAGCCGTTCTAAACTGCCGGCAAACATTGGTGTGGCCGCACAGAATTGCTATAA
AGTTGCGAAAGGCGCCTTCACCGGTGAAATTAGCCCGCAGATGATCAAAGATGTTGGCTGTG
AATGGGCAATTCTGGGTCATTCTGAACGTCGCAACGTGTTTGGCGAAAGTGATGAACTGATC
GGTGAAAAAGTTGCATTCGCGCTGGAAAGCGGCCTGAAAATTATCCCGTGCATCGGTGAAAA
ACTGGATGAACGCGAATCTGGTAAAACGGAAGAAGTGTGTTTTAAACAGCTGAAAGCCATTT
CTGATAAAGTTAGTGATTGGGATCTGGTTGTGCTGGCGTATGACCGGTGTGGGCGATTGGTA
CCGGTAAAACCGCAACGCCGGCACAGGCACAGGAAACCCACCTGGCACTGCGTAAATGGCTG
AAAGAAAACGTTAGCGAAGAAGTGTCTCAGAAAGTTCGCATTCTGTACGGCGGTAGTGTTAG
CGCGGGCAATTGCAAAGAACTGGGTACCCAGCCGGATATCGATGGCTTCCTGGTGGGTGGTG
CTTCCCTGAAACCGGACTTTGTGCAGATTATCAACGCTACGAAA
TCTP (SEQ ID NO: 26):
ATGCACCACCACCATCACCACGAAAATCTGTACTTCCAAGGCATGAAAATCTTCAAAGACGT
GTTTAGCGGCGACGAACTGTTCTCGGATACCTACAAATTTAAACTGCTGGATGATTGCCTGT
ATGAAGTGTACGGCAAATATGTTACCCGTACGGAAGGCGATGTGGTTCTGGATGGTGCGAAC
GCCAGCGCAGAAGAAGCGATGGATGATTGTGATAGCTCTAGTACCTCTGGTGTGGATGTGGT
TCTGAATCATCGCCTGGTTGAAACCGGCTTTGGTAGCAAGAAAGATTACACGGTGTATCTGA
AAGATTACATGAAGAAAGTGGTTACGTATCTGGAAGAAAACGGCAAACAGGCGGAAGTGGAT
ACCTTCAAAACGAACATCAACAAAGTTATGAAAGAACTGCTGCCGCGTTTTAAAGATCTGCA
GTTCTACACCGGTGAAACGATGGATCCGGAAGCCATGATTATCATGCTGGAATATAAAGAAG
TTGATGGCAAAGACATTCCGGTGCTGTACTTCTTCAAACACGGCCTGAACGAAGAAAAATTC

To evaluate the level of expression of our targets, small-scale cultures (4 ml) were grown to optimize the temperature, expression time, and Isopropyl β-D-1-thiogalactopyranoside (IPTG) concentration. SDS-PAGE and western blot were used to monitor expression over the different conditions. Once the optimum conditions were identified, culture volume was scaled up to 1 L to ensure ≥10 mg of protein per target.

After IPTG induction, the 1 L culture was spun down to collect cell pellets. Pellets were then lysed with lysis buffer and sonicated. Both supernatant and pellet fractions were collected and evaluated by SDS-PAGE to identify which fractions contained the target protein. For all proteins except for enolase, the proteins were located in the supernatant and therefore were soluble.

Soluble proteins were purified by adding the supernatant of the cell lysate to several millilitres of Ni-NTA (nickel-nitrilotriacetic acid) resin for high capacity, high performance nickel-IMAC (immobilized metal affinity chromatography), which is used for routine affinity purification of His-tagged proteins.

For insoluble proteins, pellets from the cell lysate were solubilized with urea, purified by N-column purification under denaturing conditions, and then refolded. Protein fractions were pooled and filter sterilized (0.22 μm).

To ensure ≥90% purity of the proteins, an additional two steps of purification by densitometric analysis of Coomassie blue stained SDS-PAGE gel was performed. Proteins were further analysed by western blot using primary mouse-anti-His mAb (GenScript, Cat. No. A00186). Protein concentration was determined using the Bradford protein assay with BSA standards (Pierce).

Aliquots were prepared in 1× PBS buffer with 10% glycerol (pH 7.4) and stored in −80° C.

The expression product of the Enolase expression DNA sequence (SEQ ID NO:22) is SEQ ID NO:27, which has the following sequence (TEV protease cleavage site is underlined, and the leading 6His tag is apparent):

MHHHHHHENLYFQGMPIKHIHARQIYDSRGNPTVEVDLTTERGIFR
AAVPSGASTGVHEALELRDKDSTWHGKSGLKAVKNVNDVLGPELVK
KNLDPVKQEEIDDFMISLDGTDNKSKFGANSILGISMAVCKAGAAH
KGVPLYRHIADLAGVKEVMMPVPAFNVINGGSHAGNKLAMQEFMIL
PTGAPSFTEAMRMGSEIYHHLKALIKKKYGLDATAVGDEGGFAPNF
QANGEAIDLLVGAIEKAGYTGKIKIGMDVAASEFYKNGKYDLDFKN
EESKEADWLTSEALGEMYKGFIKDAPVISIEDPYDQDDWEGWTALT
SQTDIQIVGDDLTVTNPKRIQMAVDKKSCNCLLLKVNQIGSVTESI
RAHNLAKSNGWGTMVSHRSGETEDCFIADLVVGLCTGQIKTGAPCR
SERLSKYNQLLRIEEELGSNAKYVGDKFRMPF

The expression product of the FBP aldolase expression DNA sequence (SEQ ID NO:23) is SEQ ID NO:28, which has the following sequence (TEV protease cleavage site is underlined, and the leading 6His tag is apparent):

MHHHHHHENLYFQGMGLEGIVPPGVITGDNLIKLFEYCRDHKVALPA
FNCTSSSTINAVLQAARDIKSPVIVQFSNGGAAFMAGKGIKNDGQKA
SVLGAIAGAQHVRLMAKHYGVPVVLHSDHCAKKLLPWFDGMLEADEE
YFKQNGEPLFSSHMLDLSEEFDEENISTCAKYFTRMTKMKMWLEMEI
GITGGEEDGVDNTNVKAESLYTKPEQVYNVYKTLSEIGPMFSIAAAF
GNVHGVYKAGNVVLSPHLLADHQKYIKEQINSPLDKPAFLVMHGGSG
STREEIAEAVSNGVIKMNIDTDTQWAYWDGLRKFYEEKKEYLQGQVG
NPEGADKPNKKFYDPRVWVRAAEESMIKRANESFESLNAVNVLGDSW
KH

The expression product of the Prx-2 expression DNA sequence (SEQ ID NO:24) is SEQ ID NO:29, which has the following sequence (TEV protease cleavage site is underlined, and the leading 6His tag is apparent):

MHHHHHHENLYFQGMSLQPTNDAPQFKAMAVVNKEFKEVSLKDYTGK
YVVLFFYPLDFTFVCPTEIIAFGDRAADFRKIGCEVLACSTDSHFSH
LHWINTPRKEGGLGDMDIPLIADKNMEISRAYGVLKEDDGVSFRGLF
IIDGTQKLRQITINDLPVGRCVDETLRLVQAFQYTDVHGEVCPAGWK
PGKKSMKPSKEGVSSYLADAEQSKK

The expression product of the TIM expression DNA sequence (SEQ ID NO:25) is SEQ ID NO:30, which has the following sequence (TEV protease cleavage site is underlined, and the leading 6His tag is apparent):

MHHHHHHENLYFQGMGGGRKFFVGGNWKMNGDKKSIDGIVDFLSKGD
LDPNCEVVVGASPCYLDYSRSKLPANIGVAAQNCYKVAKGAFTGEIS
PQMIKDVGCEWAILGHSERRNVFGESDELIGEKVAFALESGLKIIPC
IGEKLDERESGKTEEVCFKQLKAISDKVSDWDLVVLAYEPVWAIGTG
KTATPAQAQETHLALRKWLKENVSEEVSQKVRILYGGSVSAGNCKEL
GTQPDIDGFLVGGASLKPDFVQIINATK

The expression product of the TCTP expression DNA sequence (SEQ ID NO:26) is SEQ ID NO:31, which has the following sequence (TEV protease cleavage site is underlined, and the leading 6His tag is apparent):

MHHHHHHENLYFQGMKIFKDVFSGDELFSDTYKFKLLDDCLYEVYG
KYVTRTEGDVVLDGANASAEEAMDDCDSSSTSGVDVVLNHRLVETG
FGSKKDYTVYLKDYMKKVVTYLEENGKQAEVDTFKTNINKVMKELL
PRFKDLQFYTGETMDPEAMIIMLEYKEVDGKDIPVLYFFKHGLNEE
KF

The expression products were typically applied as antigens. Antigens may also be applied after 6His tag removal using TEV protease. Thus, the antigens may have a leading G residue. The variants of SEQ ID NOs:27 to 31 produced by TEV protease cleavage or as defined by SEQ ID NOs:1-6 are considered to achieve substantially the same result in substantially the same way as SEQ ID NOs:27 to 31 and as defined by SEQ ID NOs:1-6 with a leading G residue. Polynucleotide antigens encoding the same proteins are also considered to achieve substantially the same result in substantially the same way as their polynucleotide variants.

Thus, the presence or absence of a His tag or an equivalent standard tag and the present or absence of a TEV cleavage site, an equivalent cleavage site or the post-cleavage remnants thereof, are not considered to affect the antigenic properties of the protein or polynucleotide antigens.

For DNA vaccine production, each of the five antigens were cloned into the pVAX1™ plasmid vector (Invitrogen). A 3 kb vector was designed to promote high-copy number replication in E. coli and high level expression in most mammalian cell lines.

TIM was additionally cloned into the pVAC1 vector (InvivoGen). pVAC1 is a DNA vector vaccine plasmid designed to stimulate a humoral immune response via intramuscular injection. Antigenic proteins are targeted and anchored to the cell surface by cloning the gene of interest in frame upstream of the C-terminal transmembrane anchoring domain of placental alkaline phosphatase (InvivoGen). The antigenic peptide produced on the surface of muscle cells is believed to be taken up by antigen presenting cells and processed through the major histocompatibility complex class II pathway (InvivoGen).

The pVAC1-mcs backbone was selected over pVAC2-mcs for cloning because 1) the gene of interest does not contain a signal peptide even though it is secreted in vivo and 2) the vector induces a humoral immune response. The signal sequence IL-2 and the 3′ glycosyl-phosphatidylinositol (GPI) anchoring domain of human placental alkaline phosphatase directs cell surface expression of the antigenic protein (InvivoGen). The 3737 bp vector contains a Zeocin™ resistance gene and was designed for high-copy number replication in E. coli. The EF1-α gene of the pVAC1 vector ensures high levels of expression in skeletal muscle cells and antigen presenting cells. Furthermore, the SV40 enhancer gene heightens the ability of the plasmid to be transported into the nucleus, especially in non-diving cells (InvivoGen).

The vectors, pVAX1 and pVAC1, are non-fusion vectors, therefore, the inserts needed to include a Kozak translation initiation sequence (e.g. ANNATGG) containing the initiation codon and a stop codon for proper translation and termination of the gene. Primers were designed using SnapGene software to amplify a region that included the restriction enzyme site, the start codon, and the stop codon of the mRNA sequence of our target proteins. The primers are as set out in Table 3. The primers were used to amplify gene products from L. salmonis cDNA via PCR. PCR products of the expected size were PCR or gel purified, digested with the appropriate restriction enzymes, and then PCR purified again.

TABLE 3
Primers for amplification

| Protein | 5′ forward | 3′ reverse |
|---|---|---|
| FBP | GCTATCAAGCTTAAAAT | TCAGATGGATCCTTA |
| | GGGTCTTGAAGGAATTG | GTGTTTCCAGGAGTC |
| | TTC | ACCA |
| | (SEQ ID NO: 46) | (SEQ ID NO: 47) |
| Enolase | TATCGCCTGCAGAAAAT | ATCGTAGCGGCCGCT |
| | GCCTATTAAACACATTC | TAAAAGGGCATTCTG |
| | ATGCACGTC | AACTTGTC |
| | (SEQ ID NO: 44) | (SEQ ID NO: 45) |
| TIM | TAGCTGGGTACCTTACT | CGTATCAAGCTTAAA |
| | TAGTAGCGTTGATGATT | ATGGGTGGAGGAAGA |
| | TG | AAATTTTTC |
| | (SEQ ID NO: 50) | (SEQ ID NO: 51) |
| TCTP | GTCATTCTGCAGAAAAT | TCAGTAGCGGCCGCT |
| | GAAGATCTTTAAGGACG | TTTCTTCATTTAATC |
| | TAAAATTTAT | CATG |
| | (SEQ ID NO: 52) | (SEQ ID NO: 53) |
| Prx-2 | TCGACGAAGCTTAAAA | TCGACTGGTACCTTA |
| | TGAGTCTTCAACCAAC | GTTTCTTTATTGTTC |
| | GAATG | AGCATCTGCGAG |
| | (SEQ ID NO: 48) | (SEQ ID NO: 49) |

Vectors were linearized with the appropriate restriction enzymes for each insert. Linearized vector and insert were ligated with T4 DNA ligase (Invitrogen) and transformed into E. coli Stellar competent cells (Clontech). Transformants were cultured on LB plates containing 50 μg/ml kanamycin overnight at 37° C.

Single colonies were isolated and cultured overnight in 5 ml LB media+kanamycin (50 μg/ml) at 37° C. with shaking. Glycerol stocks were prepared and stored at −80° C. for each clone. Plasmid DNA was isolated from bacterial lysates using a QIAprep Spin Miniprep Kit (Qiagen) and then digested with the appropriate restriction enzymes and ran on a 1% ethidium bromide gel. Digested clones showing two bands corresponding to the size of the vector and insert were submitted for sequencing using T7 forward and BGH reverse primers (pVAX1 vector) or pVAC1 forward and pVAC1 reverse primers (pVAC1 vector)—see Table 4 for primer sequences.

TABLE 4
Primers for sequencing

| Vector | 5′ forward | 3′ reverse |
|---|---|---|
| pVAX1 | TAATACGACTC | TAGAAGGCA |
| | ACTATAGGG | CAGTCGAGG |
| | (“T7”; | (“BGH”; |
| | SEQ ID NO: 54) | SEQ ID NO: 55) |
| pVAC1 | ACTTGGTGGGTGG | AGGCACCACAGA |
| | AGACTGAAGAGT | CCTTCCAGGAT |
| | (SEQ ID NO:56) | (SEQ ID NO: 57) |

Clones containing inserts that shared high sequence similarity with the target sequence and in the correct orientation were selected for large-scale plasmid isolation. Two different kits were used for large-scale DNA vaccine preparation: Invitrogen's PureLink™ HiPure Expi Megaprep kit and Qiagen's QIAfilter plasmid giga kit. Due to the low plasmid yields obtained from the Invitrogen kit, the Qiagen giga kit was the preferred method of isolation.

A 500 ml (PureLink™ kit) or 2.5 L culture (Qiagen giga kit) was prepared following the manufacturer's instructions. Briefly, glycerol stocks of positive clones were used to streak a LB+kanamycin plate. A single colony was selected to inoculate 5 ml LB media+kanamycin and grown for 8 h at 37° C. with shaking (˜180 rpm). One milliliter was then transferred to 5-500 ml aliquots of LB media+kanamycin and grown overnight (12-14 h) for large-scale plasmid isolation the following day. All steps were performed following the manufacturer's instructions. Plasmid DNA was resuspended in nanopure water and the total amount (mg) of plasmid DNA was quantified using the NanoDrop 8000 Spectrophotometer (Thermo Scientific). Aliquots were prepared and stored at −20° C. As a quality control measure all plasmids were ran on a 1% ethidium bromide gel to check for bacterial contamination and insert. All DNA vaccines were re-sequenced before use in vaccine trial.

### Example 3

### Immunological Response to Circum-Oral Glands Peptide Recombinant Antigens

To evaluate the ability of the five candidate sea lice antigens identified in Example 1 to produce an immunological response in Atlantic salmon, the fish were vaccinated with five antigens simultaneously and the systemic antibody titer at 600 degree days after vaccination.

Treatment Groups

In more detail, Atlantic salmon of around 40 g in weight were divided into five treatment groups, each group consisting of two duplicate tanks of six salmon. The treatment groups were as follows:
1. pVAX1 vector DNA delivering all five antigens prime (i.m.; 10 μg per antigen) with subsequent i.p. boost of recombinant protein cocktail of all five antigens plus Montanide ISA 763A VG (50 μg per antigen; Delivery Method 1; “DM1”);
2. Recombinant protein cocktail of all five antigens prime plus (i.d.; 50 μg per antigen) plus flagellin (50 ng) with subsequent i.p. boost of recombinant protein cocktail of all five antigens plus Montanide ISA 763A VG (50 μg per antigen; Delivery Method 2; “DM2”);
3. Empty pVAX1 vector (i.m.) with subsequent i.p. administration of mCherry-His recombinant protein plus Montanide ISA 763A VG (“DM1 ctrl”);
4. mCherry-His prime (i.d.; 250 μg antigen) plus flagellin (50 ng) with subsequent boost of mCherry-His (i.p.; 250 μg) and
5. No vaccine control (“PBS”).

Thus, treatment groups 3 and 4 received sham treatments that contained none of the five antigens, and treatment group 5 served as a control for any non-specific immune responses to injury at vaccination of naïve fish.

The control mCherry recombinant protein was produced using the following mRNA (SEQ ID NO:32):

ATGCATCATCACCATCACCACGAAAACCTGTATTTTCAGGG
CATGGTTTCCAAAGGCGAAGAAGACAATATGGCAATCATCA
AAGAATTTATGCGTTTCAAAGTCCACATGGAAGGTTCAGTC
AATGGCCATGAATTTGAAATTGAAGGCGAAGGTGAAGGCCG
TCCGTATGAAGGTACCCAGACGGCAAAACTGAAAGTCACCA
AAGGCGGTCCGCTGCCGTTTGCTTGGGATATTCTGTCACCG
CAATTCATGTATGGTTCGAAAGCGTACGTTAAACACCCGGC
CGATATCCCGGACTACCTGAAACTGAGCTTTCCGGAAGGCT
TCAAATGGGAACGTGTTATGAACTTCGAAGATGGCGGTGTG
GTTACCGTCACGCAGGATAGCTCTCTGCAAGACGGTGAATT
CATCTACAAAGTGAAACTGCGCGGTACCAATTTCCCGTCTG
ATGGCCCGGTTATGCAGAAGAAAACCATGGGCTGGGAAGCG
AGTTCCGAACGTATGTACCCGGAAGACGGTGCCCTGAAAGG
CGAAATCAAACAGCGCCTGAAACTGAAAGATGGCGGTCATT
ATGACGCAGAAGTGAAAACCACGTACAAAGCTAAAAAACCG
GTCCAACTGCCGGGCGCATACAACGTGAACATCAAACTGGA
TATCACCAGCCACAACGAAGACTACACGATCGTTGAACAAT
ATGAACGTGCGGAAGGTCGTCACTCTACGGGCGGTATGGAT
GAACTGTACAAATAATGA

The recombinant mCherry protein had the following sequence (SEQ ID NO:33):

MHHHHHHENLYFQGMVSKGEEDNMAIIKEFMRFKVHMEGS
VNGHEFEIEGEGEGRPYEGTQTAKLKVTKGGPLPFAWDIL
SPQFMYGSKAYVKHPADIPDYLKLSFPEGFKWERVMNFED
GGVVTVTQDSSLQDGEFIYKVKLRGTNFPSDGPVMQKKTM
GWEASSERMYPEDGALKGEIKQRLKLKDGGHYDAEVKTTY
KAKKPVQLPGAYNVNIKLDITSHNEDYTIVEQYERAEGRH
STGGMDELYK

Thus, mCherry may have the sequence recited above, which has a His tag (HHHHHH; SEQ ID NO:58) and a TEV cleavage site (ENLYFQG; SEQ ID NO:59), a TEV cleaved variant sequence, or another tagged or untagged variant sequence.

A further 12 Atlantic salmon were held in duplicate tanks of 6 fish each. These fish were acclimatized for 25 days in the system prior to sampling for basal level immune responses of the population prior to vaccination. This group served as a control for basal specific antibody responses to the antigens.

All the immune sampling (i.e. blood and mucus sampling post-vaccination) occurred at 602 degree days post-priming vaccination, which the period after which you can begin to detect specific antibody titers to the vaccine antigens. Degree day was calculated by multiplying the average temperature by the number of days (DD=((T₀+T₁+ . . . )/no. of days)×no. of days).

Experimental Methods

Atlantic salmon parr approximately 40 g in weight were obtained from the USDA, Franklin, ME facility. Fish were maintained in a recirculating fresh water flow through system in 100-gallon tanks at a stocking density of 25 kg/m³ and were fed at a rate of 1.5% body weight per day. Water quality and fish condition were monitored daily.

After a 25-day acclimation period, Atlantic salmon parr were vaccinated. Atlantic salmon were anaesthetized prior to tagging and vaccination by netting fish into 100 mg/L of MS222 supplemented with 200 mg/L sodium bicarbonate as a buffer to sustain neutral pH. The fish were tagged with elastomer along the jaw line for ease of identification.

The fish were primed by intramuscular injection of the vaccine at a dose of 10 μg per antigen per fish (DM1), a cocktail recombinant protein vaccine at a dose of 50 μg per antigen per fish in a total volume of 30 μl in sterile phosphate buffered saline with 50 ng ultrapure flagellin from Pseudomonas aeruginosa (InvivoGen; “DM2”; n=48 fish per treatment group; duplicate tanks of 24 fish per group), or with the control formulation as appropriate. Post-tagging and vaccination the fish were returned to their respective housing tanks and monitored continuously until full recovery.

Two weeks after prime vaccination, the fish were anaesthetized and received a boost vaccination of recombinant proteins intraperitoneally at a dose of 50 μg per protein per fish, adjuvanted with Montanide™ ISA 763 A VG in a total volume of 100 μl (DM1 and DM2).

To measure the specific antibody response to louse antigens post-vaccination the blood and mucus of 12 Atlantic salmon per treatment group were sampled at 602-degree days for ELISA and dot blot analysis, respectively. Fish were euthanized with a lethal dose of 250 mg/L MS-222 buffered with 100 mg/L sodium bicarbonate. Blood was collected by bleeding the fish via the caudal vein. Blood samples were incubated at 4° C. overnight and serum was isolated by centrifugation at 3716×g for 10 min at 4° C. Serum was isolated and stored at −80° C. until further use. Skin mucus samples were collected by placing the fish in a bag containing 10 ml phosphate buffered saline and massaging the fish for 2 minute each to wash off mucus. Mucus was centrifuged at 3716×g for 10 minutes at 4° C. and the supernatant transferred into sterile tubes and stored at −80° C.

The efficacies of the vaccines in eliciting a systemic immune response were evaluated for each vaccine candidate. All ELISA's were optimized prior to running serum samples from each fish. Optimal protein concentration, primary, and secondary antibody concentrations were determined for each antigen by running a checkerboard assay (Table 5).

TABLE 5
Checkerboard assay results for antibody
detection of sea louse antigens.

| Protein | Stock μg/ml | Coating | Primary | Secondary |
|---|---|---|---|---|
| TIM | 12600 | 2 μg/ml | 1/500 | 1/500 |
| FBP | 12500 | 2 μg/ml | 1/1000 | 1/1000 |
| Prx-2 | 11800 | 2 μg/ml | 1/1000 | 1/2000 |
| TCTP | 17900 | 2 μg/ml | 1/500 | 1/1000 |
| Enolase | 620 | 2 μg/ml | 1/500 | 1/2000 |

One hundred microliters of antigen (2 μg per well in carbonate:bicarbonate coating buffer; Sigma) was coated onto the wells of a 96-well polystyrene microtiter plate (Thermo Scientific). Plates were washed with low salt wash buffer (3×) and then blocked overnight at 4° C. with 3% (w/v) casein in deionized water. After three more washes with low salt wash buffer, serum dilutions (1/100) in PBS were added to each well and allowed to incubate overnight at 4° C. (100 μl per well). Plates were washed 5× with high salt wash buffer to remove residual serum and unbound antibodies. Primary antibody, mouse anti salmonid Ig monoclonal (Biorad; cat #MCA2182), was diluted to the appropriate concentration in PBS (Table 5) and added to each well (100 μl/well) and incubated at room temperature for 1 h. Plates were washed with high salt wash buffer (5×) to remove unbound antibody. The secondary antibody, goat anti-mouse IgG peroxidase (Sigma; cat #A4416), was diluted to the appropriate concentration with conjugate buffer (1% (w/v) bovine serum albumin diluted in low salt wash buffer) and added to the wells. After a 1 hr incubation at room temperature followed by 5× wash with high salt wash buffer, 100 μl of the chromogen (TMB) was added to each well and incubated for 10 min at room temperature. The reaction was stopped by adding 50 μl 2 M sulfuric acid to each well. Plates were mixed and the absorbance was recorded at 450 nm using a spectrophotometer. Each plate contained relevant controls: 1) pooled positive serum, 2) pooled negative serum, and 3) no serum controls (PBS). The coefficient of variation of the A450 nm of sample replicates within a plate, and the pooled positive serum between plates was always ≤20%.

Results

At 602 degree days after vaccination, Atlantic salmon serum antibody levels were measured to the five sea louse antigens included in the vaccine. ELISA analysis data showed Atlantic salmon responded to all five antigens delivered in the cocktail vaccine with a DNA prime (FIGS. 1-5), or a recombinant protein prime (FIGS. 6-10).

An immunological response was also induced by prime vaccination with 10 μg TIM DNA antigen either in a pVAX1 vector or a pVAC1 vector, following by a boost using 50 μg of TIM recombinant protein.

Thus, TIM, FBP, Prx-2, TCTP and Enolase each provides an antigen that elicits an immunogenic response in fish.

### Example 4

### Efficacy of Sea Lice Vaccine Candidates

The efficacy of sea lice vaccine candidates against Lepeophtheirus salmonis (salmon louse) infection in Atlantic salmon (Salmo salar) was evaluated.

The specific antibody response was measured across 6 treatments (n =15 fish per treatment). Controls included a control for the His-tag as well as a no injection control (phosphate buffered saline [PBS]). The His-tag control served as a control for the His tag on the bacterially expressed sea louse antigens. PBS served as a control for any non-specific immune responses to injury at vaccination and to allow for the evaluation of sea lice settlement of non-vaccinated fish. An additional 42 fish per treatment were vaccinated and sampled to measure vaccine efficacy post sea lice challenge.

Treatments:
Vaccine 1: enolase (SEQ ID NO:1)
Vaccine 2: Prx-2 (SEQ ID NO:4)
Vaccine 3: TIM (SEQ ID NO:5)
Vaccine 4: FBP (SEQ ID NO:3)
Vaccine 5: TCTP (SEQ ID NO:6)
Vaccine 6: vehicle control (phosphate buffered saline—PBS)

For the prime vaccination, each recombinant protein vaccine contained 100 ng of purified flagellin from Pseudomonas aeruginosa (FLA-PA Ultrapure, InvivoGen) and was adjuvanted (Montanide™ ISA 763 A VG; Seppic™). For the boost vaccination, each vaccine formulation was adjuvanted (Montanide™ ISA 763 A VG; Seppic™)

Vaccine Production

Recombinant protein vaccines were prepared by inoculating lysogenic broth (LB)-kanamycin (50 μg) agar plates with glycerol stocks of E. coli BL21 (DE3) cells, which contain the pET-30a (+) expression plasmid (Novagen) with gene insert, and growing each vaccine candidate overnight at 37° C. Single colonies were isolated and used to inoculate 2-50 ml flasks of LB with kanamycin (50 μg). Cultures were allowed to grow at 37° C. with shaking for 2-4 hours or until the optical density at 600 nm was reached (0.6 to 0.8). Approximately 16.6 ml of culture media was added to 500 ml of LB with kanamycin (50 μg) in a flask for overnight growth at 200 rpm and 37° C. Once target optical densities were reached (i.e. 0.6 to 0.8), IPTG was added at 1 mM dose to each 500 ml flask and temperature was reduced to 18° C. with shaking at 200 rpm. After 15-18 hr of induction, the optical density was measured (target optical densities of 1-7) and cultures were centrifuged at 10,000×g for 10 min at 4° C. The weight of each pellet was measured in each centrifugation bottle. Based on that weight, the amount of lysis buffer was calculated (2 ml of lysis buffer per 100 mg of cell pellet), and pellets were resuspended with vortexing. DNase was added (2 U per ml of lysis buffer) to each bottle and mixed gently. Pellets were sonicated on ice in 20 second bursts for a total of 4 min and then incubated on ice for 15 min with intermittent mixing followed by centrifugation for 20 min at 10,000×g at 4° C. The supernatant was decanted and added to a nickel-iminodiacetic acid-based protein purification resin (His60 Ni Superflow Resin; Takara), and allowed to incubate for 2 to 24 hours with gently stirring at 4° C.

Some proteins (e.g. Prx-2) were shown to have a high affinity for the resin and therefore lower incubation times were preferred (˜2 h). Lower affinity proteins (e.g. FBP and TCTP) were allowed to mix with the resin for at least 24 h. Resin and supernatant (˜250-300 ml) was added to 4-10 ml polypropylene gravity flow purification columns (Thermo Scientific, catalog #29924). Once the resin settled to the bottom of the column, 10 ml of equilibration buffer was added (×2). This was followed by 10 ml of wash buffer (×2). The protein was eluted from the column by adding multiple 10 ml aliquots of elution buffer until protein detection by 280 nm light absorbance was negligible. For high affinity proteins, elution buffer containing 400 mM imidazole was added. For lower affinity proteins, 300 mM imidazole elution buffer was used. The eluate for each protein was combined and concentrated using 20 ml, 5 kDa, MWCO concentrators (GE Healthcare catalog #28-9329-59). Excess imidazole was removed by adding concentrates to PD-10 desalting columns (GE Healthcare). Protein was concentrated briefly again and then filter sterilized with 0.22 13 mm diameter, PVDF syringe filters (Celltreat® catalog #229742). A sterile 80% glycerol solution was added to each protein aliquot to give 8-10% glycerol per tube prior to storage at −80° C. (Acros Organics CAS 56-81-5). Protein concentration was determined using a Pierce® BCA Protein Assay Kit (Thermo Scientific catalog #23227). Proteins that were difficult to express at the quantities required (e.g. enolase) were produced by enhanced methods known to the skilled person (GenScript® protein expression service).

Atlantic Salmon

Atlantic salmon post smolts (n=342) approximately 70 to 100 grams in weight were maintained in a recirculating artificial salt water system on a 12:12 hr light:dark cycle in 100-gallon tanks at a stocking density of 25 kg/m³. Water quality, ammonia, nitrite, and fish condition were monitored daily. Salmon were fed a daily ration of BioTrout 3 mm pellets (Bio-Oregon®) at 1.5% body weight per day and maintained at temperatures of 13±1° C., 32±1% salinity, and 8±1 mg/L dissolved oxygen (means±standard deviations).

Fish Vaccination

Fish size ranged from 98 to 295 grams at prime vaccination (average size 180 g). There were two vaccine treatments per tank (n=19 fish per antigen) in replicates of three tanks (n=38 fish per tank). During the vaccination phase, fish stocking density was ≤18.1 kg/m³. Prior to vaccination, 20 fish were euthanized for mucus and blood collection with a lethal dose of MS-222 (250 mg/L). These fish served as a measure of the basal level of immunity of the fish. Fish to be vaccinated were anaesthetized with 100 mg/L MS-222 and then primed intradermally using a sterile 25-gauge needle and syringe. A 200 μg dose was prepared for the following recombinant proteins: enolase, Prx-2, TIM, FBP and TCTP (n=57 fish per treatment). The number of injections per antigen ranged between two to three 10-μl injections per fish to achieve the target dose. For the PBS control, a single 10 μl dose was injected into each fish (n=57). To distinguish fish between vaccine groups, an elastomer tag (Northwest Marine Technology, Inc.) was injected under the skin along the jawline following the intradermal injection of antigen. Each recombinant protein vaccine contained 100 ng of FLA-PA Ultrapure flagellin from P. aeruginosa (InvivoGen cat #tlrl-pafla). Each vaccine formulation was adjuvanted with Montanide™ ISA 763 A VG (Seppic™). Once primed, fish were returned to their respective treatment tanks to recover.

Two weeks post-prime vaccination, fish were anesthetized and boosted with an intraperitoneal (i.p.) injection of the recombinant protein vaccines, except for Prx-2 proteins, which was boosted 3 weeks and 4 days post-prime vaccination (n=11). One hundred microliters of a 200 μg dose was prepared for the following recombinant proteins: enolase, Prx-2, TIM, FBP and TCTP (n=57 fish per treatment). Each vaccine formulation was adjuvanted with Montanide™ ISA 763 A VG (Seppic™ Lot #36017Z). One hundred microliters of antigen at the described doses (above) plus Montanide was i.p. injected into each fish. For the PBS control, 100 μl PBS was added with adjuvant. Once boosted, fish were returned to their respective treatment tanks to recover.

At least three weeks prior to sea lice challenge, Atlantic salmon approximately 240 g in size were cohabitated into eight replicate tanks. Around 5 fish per treatment were transferred into each tank giving a total of 65 fish per tank or a stalking density of 41.3 kg/m³.

Serum and Mucus Collection

At 602 degree days, 43 days after boost vaccination and 588 degree days (42 days after boost), 15 fish per treatment were euthanized by exposing fish to an overdose of M-5222 (250 mg/L) to measure specific antibody responses after vaccination (n=90 fish). Serum was collected by bleeding the fish via the caudal vein using a sterile 23-gauge needle with a 3 ml syringe. Samples were processed by incubating samples at 4° C. overnight and then centrifuging the blood at 3000×g for 10 min at 4° C. The supernatant containing the plasma was collected and transferred into 2-1.5 ml microcentrifuge tubes and stored at −80° C. for ELISA analysis. Skin mucus samples were collected by placing each fish into a bag containing 10 ml phosphate buffered saline and massaging the fish for 2 minute each to wash off mucus. Samples were centrifuged for 15 minutes at 1500×g at 4° C. Mucus was transferred into two 1.5 ml microcentrifuge tubes and stored at −80° C. for dot blot analysis.

L. salmonis Challenge

Two thousand L. salmonis egg strings were collected from gravid females and transferred to a sea lice hatchery. L. salmonis copepodids of similar age (3-4 days old) were pooled and the number of copepodids were calculated by counting ten 1-ml aliquots of lice using a dissecting scope to give the mean number of copepodids per ml of seawater. Infections were performed by reducing the volume of the tank holding the fish to a third of the original volume and copepodids were added to each of the replicate tanks to give an infection density of 80 copepodids per fish. The dissolved oxygen was monitored continuously throughout the 1-hour bath infection to maintain dissolved oxygen at 8.5±1.0 mg/L (mean±standard deviation). After one hour, the tank water level was restored. Dissolved oxygen was monitored for another 1.5 hours before turning the flow back on to each tank. Fish were monitored for an additional hour to ensure dissolved oxygen and flow rate were maintained in each tank at the appropriate levels.

To evaluate vaccine efficacy against salmon louse attachment, Atlantic salmon (n=42 fish per treatment; n=252) were challenged with L. salmonis copepodids 980 or 994 degree days after boost vaccination). Eight to eleven days after sea lice challenge, the salmon were exposed to an overdose of MS-222 to perform sea lice counts. Blind counts of the chalimus stages were recorded from the skin and gills of each fish for each treatment using a dissecting microscope and forceps. To reduce count variation, the same four individuals manually counted the number of lice on each fish. After counts were completed, the length (mm) and weight (g) of each fish was recorded.

Data Analysis

The relative intensity (RI), which is the total number of lice per gram body weight [RI=total number lice/total weight (g)], was calculated for each individual fish subject to a vaccine treatment (Myksvoll et al., 2018). The RI values between vaccine treatments were compared. The average relative intensity (ARI=average number of lice/average weight [g]) was calculated to determine the percent change in lice intensity between vaccinated treatments and the PBS control (Myksvoll et al., 2018). Using these values, the % change was calculated (ARI PBS control−ARI vaccine antigen)/(ARI PBS control)×100).

Results

The data from the sea lice vaccine trial showed that vaccination with recombinant protein antigens identified from the circum-oral glands of the chalimus stages reduced the number of chalimus per fish caused by the sea lice challenge.

Prx-2 and FBP were shown to be the most protective of the tested antigens, as shown in the RI values reported in Table 6.

TABLE 6
Mean relative intensity of sea lice post vaccination
and challenge with L. salmonis.

| Antigen | RI (mean ± SEM) |
|---|---|
| PBS control | 0.164 ± 0.016 |
| Prx-2 | 0.114 ± 0.012 |
| FBP | 0.117 ± 0.014 |
| TCTP | 0.125 ± 0.010 |
| Enolase | 0.132 ± 0.013 |
| TIM | 0.135 ± 0.010 |

The percent reductions in chalimus counts for Prx-2, FPB, enolase, TCTP, and TIM were 28.9% to 13.1% (Table 7).

TABLE 7
Percent reduction of L. salmonis chalimus stages after vaccination.

| Antigen | Reduction (%) |
|---|---|
| Prx-2 | 28.9 |
| FBP | 25.1 |
| Enolase | 19.1 |
| TCTP | 17.9 |
| TIM | 13.1 |

Atlantic salmon were vaccinated with 5 different L. salmonis candidate antigens and challenged with the infective stage of the parasite. Using the average relative intensity, the percent change between the PBS control and candidate vaccine was calculated.

The antigens (FBP, TCTP, TIM, Prx-2, and enolase) had no negative effect on the growth of the vaccinated fish. Thus, vaccination with the L. salmonis antigens identified from the circum-oral glands of the chalimus stages reduced the relative intensity of chalimus infestation on Atlantic salmon.

The immunogenicity of the candidate antigens was assessed by western blot. Data showed that the pooled serum samples from vaccinated and sea lice challenged fish contained antibodies to the sea lice vaccine antigens. Protein bands of the correct sizes were detected on the nitrocellulose membrane after development (FBP, 42.1 kDa; TCTP, 21.6 kDa; enolase, 48.9 kDa; TIM, 28.7 kDa; and Prx-2, 24.0 kDa). These results suggest that the monovalent recombinant protein vaccines, Prx-2, FBP, Enolase, TIM, and, TCTP induced an antibody response in the host. Furthermore, the results show that the antigenic response to the vaccines by the host was protective upon secondary challenge with sea lice.

### REFERENCES

Bommer and Thiele, 2004. International J Biochem & Cell Biol 36(3), 379-385.

Denholm, 2002. Pest Manag Sci 58: 528-536.

Díaz-Ramos et al, 2012. Biotech, Article ID 156795,12 pages. doi:10.1155/2012/156795.

Donnelly et al., 2008. The FASEB Journal, 22(11), 4022-4032.

El Ridi et al., 2013. J Parasitol 99,194-202.

Fast et al., 2003. J Parasitol 89: 7-13.

Fast et al., 2004. Exp Parasitol. 107:5-13.

Furuya and Ikeda 2011. Microbiol Immunol 55,855-862.

Gnanasekar et al, 2002. Molecular Biochem Parasitol 121 (1), 107-118

Gnanasekar and Ramaswamy, 2007. Parasitol Res 101(6), 1533-1540.

Gnanasekar et al., 2009. Biochem Biophysical Res Comm, 386(2), 333-337.

Gonzalez and Carvajal, 2003. Aquaculture 220: 101-117.

Grimnes et al., 1996. J Fish Biol 48: 1179-1194.

Hu et al., 2015. Parasitol. Res. 114 (9), 3459-68.

Johnson et al, 2004. “Interactions between sea lice and their hosts”. In: Host-Parasite Interactions. Editors: G. Wiegertjes and G. Flik, Garland Science/Bios Science Publications, pp. 131-160.

Jones et al., 1990. J Fish Dis 13: 303-310.

Jónsdóttir et al., 1992. J Fish Dis 15: 521-527.

Kabata, 1970. Book 1: Crustacea as enemies of fishes. In: Diseases of Fishes. Editors: Snieszko, S. F. and Axelrod, H. R.; New York, T.F.H. Publications, p. 171.

Knoops et al., 2016. Mol. Cells 39,60.

Lorenzatto et al., 2012. Gene, 506(1), 76-84.

MacKinnon, 1997. World Aquaculture 28: 5-10.

McCarthy et al., 2002. Infection and Immunity 70(2): 851-858.

Meyvis et al., 2009. International J. Parasitology 39(11), 1205-1213.

Myksvoll M S et al., 2018. PLoS ONE 13(7): e0201338.

Nagano-Ito et al., 2009. FEBS Letters 583 (8), 1363-1367.

Nagano-Ito et al., 2012. Biochem Res. International 2012, 204960.

Pal-Bhowmick et al., 2007. Malaria Journal 6 (45), 1-7.

Pereira et al., 2007. FEMS Yeast Res 7,1381-1388.

Pike et al., 1999. Adv Parasitol 44: 233-337.

Rhee et al., 2016. Molecules and Cells 39(1), 1-5.

Saber et al., 2013. Korean J of Parasitol 51(2), 155-163.

Sangpairoj et al., 2014. Exp Parasitol 140,24-32.

Saramago et al., 2012. International J Mol Sci 13(10), 13118-13133.

Schram, 1993. “Supplemental descriptions of the developmental stages of Lepeophtheirus salmonis (Kroyer, 1837) (Copepoda: Caligidae)”. In: Pathogens of Wild and Farmed Fish: Sea Lice. Editors: Boxshall, G. A. and Defaye, D., pp. 30-50.

Stone et al., 1999. J Fish Dis 22: 261-270.

Sun et al., 2008. Biochimie 90(11-12), 1760-1768.

Tsuji et al. 2001. Insect Mol Biol 10: 121-129.

Walker et al., 2000. Antimicrobial Agents and Chemotherapy 44,344-347.

Wang et al., 2013. PLoS ONE 8(7), e69284.

Wood et al., 2003. Trends Biochem Sci 28,32-40.

Wikel et al., 1996. “Arthropod modulation of host immune responses”. In The Immunology of Host-Ectoparasitic Arthropod Relationships. Editors: Wikel, S. K., CAB Int., pp. 107-130.

Zhu et al., 2004. Southeast Asian J Trop Med Public Health 35(3): 518-522.

1. An antigen comprising one or more isolated protein, which is isolated from the circum-oral gland (COG) or the frontal gland complex (FGC) of a caligid copepod, wherein the protein is selected from the group consisting of: peroxiredoxin-2 (Prx-2), fructose bisphosphate aldolase (FBP), enolase, transitionally-controlled tumour protein homolog (TCTP), and triosephosphate isomerase (TIM); optionally wherein the caligid copepod is Lepeophtheirus salmonis or Caligus rogercresseyi.

2.-3. (canceled)

4. The antigen according to claim 1, comprising the amino acid sequence of the one or more protein is selected from the group consisting of: SEQ ID NO:4; SEQ ID NO:3; SEQ ID NO:1; SEQ ID NO:2; SEQ ID NO:6; SEQ ID NO:5; and homologues thereof.

5.-6. (canceled)

7. A vaccine against caligid copepod infection in fish, the vaccine comprising an immunologically effective amount of one or more antigen according to claim 1, and a pharmaceutically-acceptable diluent or carrier, and optionally an adjuvant, optionally wherein the caligid copepod is Lepeophtheirus salmonis or Caligus rogercresseyi.

8. A vaccine according to claim 7, wherein each of the one or more antigens is different from the other antigen or antigens in the vaccine.

9. A vaccine according to claim 8, wherein the vaccine comprises five antigens, wherein one of the five antigens comprises FBP, one of the five antigens comprises TIM, one of the five antigens comprises Prx-2, one of the five antigens comprises enolase, and one of the five antigens comprises TCTP.

10. A vaccine according to claim 8, wherein the vaccine comprises five antigens, wherein one of the five antigens comprises the amino acid sequence of SEQ ID NO:1 or SEQ ID NO:2 or homologues thereof, one of the five antigens comprises the amino acid sequence of SEQ ID NO:3 or homologues thereof, one of the five antigens comprises the amino acid sequence of SEQ ID NO:4 or homologues thereof, one of the five antigens comprises the amino acid sequence of SEQ ID NO:5 or homologues thereof, and one of the five antigens comprises the amino acid sequence of SEQ ID NO:6 or homologues thereof.

11. (canceled)

12. The vaccine according to claim 7, wherein the fish is a salmonid.

13.-17. (canceled)

18. An antigen comprising a polynucleotide comprising DNA encoding a protein isolated from the circum-oral gland (COG) or the frontal gland complex (FGC) of a caligid copepod, wherein the protein is selected from the group consisting of: peroxiredoxin-2 (Prx-2), fructose bisphosphate aldolase (FBP), enolase, transitionally-controlled tumour protein homolog (TCTP), and triosephosphate isomerase (TIM); optionally wherein the caligid copepod is Lepeophtheirus salmonis or Caligus rogercresseyi.

19.-20. (canceled)

21. The antigen according to claim 18, wherein the polynucleotide comprises DNA encoding the amino acid sequence of one or more of the group consisting of: SEQ ID NO:4; SEQ ID NO:3; SEQ ID NO:1; SEQ ID NO:2; SEQ ID NO:6; SEQ ID NO:5; and homologues thereof.

22. The antigen according to claim 18, wherein the DNA comprises the nucleotide sequence of one or more of the group consisting of: SEQ ID NO:12; SEQ ID NO:11; SEQ ID NO:10; SEQ ID NO:9; SEQ ID NO:8; SEQ ID NO:7; SEQ ID NO:16; SEQ ID NO:15; SEQ ID NO:14; SEQ ID NO:13;and homologues thereof.

23. (canceled)

24. A vaccine against caligid copepod infection in fish, the vaccine comprising an immunologically effective amount of one or more antigen according to claim 18, a pharmaceutically-acceptable diluent or carrier, and optionally an adjuvant.

25. The vaccine against caligid copepod infection in fish according to claim 24, wherein the vaccine comprises an immunologically effective amount of a combination of two or more antigens, wherein each of the one or more antigens independently comprises the DNA sequence selected from the group consisting of: SEQ ID NO:12; SEQ ID NO:11; SEQ ID NO:10; SEQ ID NO:9; SEQ ID NO:8; SEQ ID NO:7; SEQ ID NO:16; SEQ ID NO:15; SEQ ID NO:14; SEQ ID NO:13; and homologues thereof.

26. The vaccine according to claim 24, wherein each of the one or more antigens is different from the other antigen or antigens in the vaccine.

27. The vaccine according to claim 26, wherein the vaccine comprises five antigens, wherein one of the five antigens comprises the DNA sequence of SEQ ID NO:7 or SEQ ID NO:8 or homologues thereof, one of the five antigens comprises the DNA sequence of SEQ ID NO:9 or SEQ ID NO:10 or homologues thereof, one of the five antigens comprises the DNA sequence of SEQ ID NO:11 or SEQ ID NO:12 or homologues thereof, one of the five antigens comprises the DNA sequence of SEQ ID NO:13 or SEQ ID NO:14 or homologues thereof, and one of the five antigens comprises the DNA sequence of SEQ ID NO:15 or SEQ ID NO:16 or homologues thereof.

28. (canceled)

29. The vaccine according to claim 24, wherein the fish is a salmonid.

30.-34. (canceled)

35. A method of treatment or prevention of caligid copepod infection in fish, comprising administering a therapeutic amount of the antigen of claim 1, optionally with the co-administration of an adjuvant, optionally wherein the caligid copepod is Lepeophtheirus salmonis or Caligus rogercresseyi.

36. (canceled)

37. The method according to claim 35, wherein the fish is a salmonid.

38. (canceled)

39. A method of treatment or prevention of caligid copepod infection in fish, comprising administering a therapeutic amount of the antigen of claim 18, optionally with the co-administration of an adjuvant, optionally wherein the caligid copepod is Lepeophtheirus salmonis or Caligus rogercresseyi.

40. The method according to claim 39, wherein the fish is a salmonid.
